(19) United States
(12) Patent Application Publication
(10) Pub. No.: US 2026/0000470 A1
(43) Pub. Date: Jan. 1, 2026

(54) ROBOTIC SURGERY SYSTEM WITH SURGICAL VISUALIZATION

(71) Applicant: MAKO Surgical Corp., Weston, FL (US)

(72) Inventors: Milan Ikits, Weston, FL (US); Ajeet Singh Yadav, Weston, FL (US)

(21) Appl. No.: 19/238,879

(22) Filed: Jun. 16, 2025

Related U.S. Application Data
(60) Provisional application No. 63/664,847, filed on Jun. 27, 2024.

(51) Int. Cl. A61B 34/30; A61B 17/16; A61B 34/00; A61B 34/10; A61B 34/20

(52) U.S. Cl. CPC A61B 34/30; A61B 17/1615; A61B 17/1684; A61B 34/10; A61B 34/20; A61B 34/25; A61B 2034/104; A61B 2034/105; A61B 2034/107; A61B 2034/108; A61B 2034/2055; A61B 2034/254

(57) ABSTRACT

A method includes providing a bone model and a surgical plan defining a planned implant pose to the bone model, providing, based on the planned implant pose, a planned resection volume and a cut-away object, generating a visualization for surgical navigation by, subtracting, from the bone model, a region of intersection between the bone model and the cut-away object and including a visualization of the planned resection volume, and dynamically updating the visualization of the planned resection volume to show progress in completing a resection by tracking movement of a surgical instrument completing the resection.

### CROSS-REFERENCE TO RELATED APPLICATION

This application claims the benefit of and priority to U.S. Provisional Patent Application No. 63/664,847, filed Jun. 27, 2024, the entire disclosure of which is incorporated by reference herein.

### BACKGROUND

The present disclosure generally relates to methods and systems for providing a visual representation of a cut procedure and, more specifically, to computer-implemented methods and systems for providing a visual representation of material removal from an object during a cut procedure.

The transformation of a cut procedure into a visual representation is of interest in many applications such as surgery, machining, and milling. For example, there is an interest in developing and improving systems and methods for providing a visual representation of surgical resection procedures as a cutting tool (e.g., burr, saw, reamer, etc.) is penetrating the bone or tissue of a patient. More particularly, providing real time (or perceived real time) updates of the progress of bone or tissue removal at a display screen allows a surgeon or other medical staff to visualize material removal from the bone or tissue as the cutting tool penetrates the bone or tissue.

Visualization of a resection procedure can be computationally expensive, in some cases resulting in delays that provide a lag between actual progress of bone or tissue removal and graphical updates at a display screen. Additionally, even if a cutting tool travels along a smooth, continuous path during the resection procedure, visualizations can often have visual artifacts such as edgy, ridge-like patterns that do not accurately represent resected surfaces. Additionally, three-dimensional resections can be difficult to visualize efficiently and clearly on a two dimensional display.

Thus, innovations for providing a visual representation of material removal during a cut procedure, such as a bone resection procedure, which reduce computational expense while reducing visualization artifacts are desirable.

### SUMMARY

One implementation of the present disclosure is a method. The method includes providing a virtual bone model and a surgical plan defining a planned cut relative to the virtual bone model, orienting a voxel grid relative to the bone model based on the planned cut, obtaining tracking data indicative of positions of a cutting tool, and providing guidance for execution of the planned cut by generating a visualization based on the voxel grid and the tracking data.

Another implementation of the present disclosure is one or more non-transitory computer-readable media storing program instructions that, when executed by one or more processors, cause the one or more processors to perform operations. The operations include providing a bone model and a surgical plan defining a planned cut relative to the virtual bone model and orienting the bone model relative to a graphics coordinate system based on the planned cut such that the planned cut is orthogonal to a first dimension of the graphics coordinate system, parallel to a second dimension of the graphics coordinate system, and parallel to a third dimension of the graphics coordinate system. The operations also include generating a visualization of execution of the planned cut based on the graphics coordinate system, the bone model, and tracking data indicative of positions of a cutting tool during the execution of the planned cut.

Another implementation of the present disclosure is a surgical system. The surgical system includes a tool operable to modify a bone. The surgical system also includes circuitry programmed to orient a voxel grid relative to a model of the bone based on a planned bone modification and generate, using the voxel grid, a visualization of progress of the tool in executing the planned bone modification based on tracking data indicative of positions of the tool.

Another implementation of the present disclosure is a method. The method includes providing a bone model and a surgical plan defining a planned implant pose to the bone model, providing, based on the planned implant pose, a planned resection volume and a cut-away object, generating a visualization for surgical navigation by subtracting, from the bone model, a region of intersection between the bone model and the cut-away object and including a visualization of the planned resection volume, and dynamically updating the visualization of the planned resection volume to show progress in completing a resection by tracking movement of a surgical instrument completing the resection.

In some embodiments, providing the planned resection volume and the cut-away object includes retrieving the planned resection volume and the cut-away object from a library based on a selected size of an implant to be implanted in the planned implant pose. In some embodiments, the method includes orienting the cut-away object relative to the planned resection volume based on an anatomic axis or plane of the bone model. In some embodiments, dynamically updating the visualization of the planned resection volume includes providing a remainder of the visualization of the planned resection volume to be resected in a first color, coloring an over-resected region beyond the planned resection volume in a second color, and providing a resected surface between the over-resected region and the visualization of the planned resection volume with a color gradient from the first color to the second color.

In some embodiments, the method also includes including, with the visualization, a virtual model of the surgical instrument by coloring the surgical instrument in a first color prior to alignment with the planned resection volume, coloring the surgical instrument in a second color while aligned with the planned resection volume, and coloring the surgical instrument in a third color when the surgical instrument reaches a boundary of the planned resection volume or deviates from the planned resection volume.

Another implementation of the present disclosure is a computer visualization method for surgical planning and/or navigation. The computer visualization method includes grouping constructive solid geometry (CSG) objects associated with at surgical scene into a first group and a second group, performing a first CSG operation using CSG objects in the first group independent of CSG objects in the second group, performing a second CSG operation using CSG objects in the second group independent of CSG objects in the first group, and visualizing a combined CSG scene using a first result of the first CSG operation and a second result of the second CSG operation.

### BRIEF DESCRIPTION OF THE FIGURES

FIG. 1 is a perspective view of a system configured to provide a visual representation of material removal from an object by a cutting tool during a cut procedure, constructed in accordance with the present disclosure.

FIG. 2 is a flowchart of a series of steps involved in generating the visual representation of the cut procedure, in accordance with a method of the present disclosure.

FIG. 3 is a schematic representation of the concept of shape sweeping of consecutive poses of the cutting tool, in accordance with the present disclosure.

FIG. 4 is a schematic representation of a CSG operation of subtraction, in accordance with the present disclosure.

FIG. 5 is a schematic representation of a CSG operation of union, in accordance with the present disclosure.

FIG. 6 is a schematic operation of a CSG operation of intersection, in accordance with the present disclosure.

FIG. 7 is a flowchart of steps involved in generating an empty voxelized constructive solid geometry (CSG) grid for accumulation of the union of the cut trajectory, in accordance with a method of the present disclosure.

FIG. 8 is a flowchart of a series of steps that may be involved in the method of FIG. 2, in accordance of a method of the present disclosure.

FIG. 9 is a flowchart of a series of steps that may be involved in performing CSG compositing, in accordance with the method of FIG. 2.

FIG. 10 is a schematic representation of the concept of defining an active ray range for non-polygonal iso-surface ray marching, in accordance with a method of the present disclosure.

FIG. 11 is a schematic representation of non-polygonal iso-surface ray marching to compute a subtraction of the union of the cut trajectory from the object, in accordance with a method of the present disclosure.

FIG. 12 is schematic representation of subtracting an un-swept cut trajectory from the object, in accordance with the prior art.

FIG. 13 is a schematic representation of subtracting a swept cut trajectory from the object to produce smooth cut surfaces in the visual representation, in accordance with the present disclosure.

FIG. 14 is a flowchart of a plan-dependent visualization process, according to some embodiments.

FIG. 15 is an illustration of a bone model, cut plane, and bounding box as used in some examples of the plan-dependent visualization process of FIG. 14, according to some embodiments.

FIG. 16 is a schematic illustration relating to the plan-dependent visualization process of FIG. 14, according to some embodiments.

FIG. 17 is another schematic illustration relating to the plan-dependent visualization process of FIG. 14, according to some embodiments.

FIG. 18 is an illustration showing results of applying the plan-dependent visualization process of FIG. 14 as compared to another approach, according to experimental results in some embodiments.

FIG. 19 is a flowchart of another plan-dependent visualization process, according to some embodiments.

FIG. 20 is an illustration showing results of applying the plan-dependent visualization process of FIG. 19, according to some embodiments.

FIG. 21 is an illustration of use of procedural geometry, according to some embodiments.

FIG. 22 is a flowchart of a process for generating a visualization using procedural geometry, according to some embodiments.

FIG. 23 is another flowchart relating to generating a visualization using procedural geometry, according to some embodiments.

FIG. 24 is a flowchart of a process for providing visualizations with transparency, according to some embodiments.

FIG. 25 is a diagram illustrating features for providing visualizations with transparency, according to some embodiments.

FIG. 26 is a flowchart of a process for providing a color gradient using a transparency technique, according to some embodiments.

FIG. 27 is a diagram of options for changing color of a visualization proximate a boundary of a planned resection volume, according to some embodiments.

FIG. 28 is a flowchart of a process for providing a visualization of multiple objects, according to some embodiments.

FIG. 29 is a diagram illustrating grouping of visualization operations for example in accordance with the process of FIG. 28, according to some embodiments.

FIG. 30 is an example implementation of the grouping technique of FIGS. 28-29, according to some embodiments.

FIG. 31 is another example implementation of the grouping technique of FIGS. 28-29, according to some embodiments.

FIG. 32 is a flowchart of a process for providing a visualization for a three-dimensional resection including transparent and/or cut-away visualizations, according to some embodiments.

FIG. 33 is a first example graphical user interface that can be provided by the process of FIG. 32, according to some embodiments.

FIG. 34 is a second example graphical user interface that can be provided by the process of FIG. 32, according to some embodiments.

FIG. 35 is a third example graphical user interface that can be provided by the process of FIG. 32, according to some embodiments.

FIG. 36 is a fourth example graphical user interface that can be provided by the process of FIG. 32, according to some embodiments.

FIG. 37 is a fifth example graphical user interface that can be provided by the process of FIG. 32, according to some embodiments.

FIG. 38 is a sixth example graphical user interface that can be provided by the process of FIG. 32, according to some embodiments.

FIG. 39 is a seventh example graphical user interface that can be provided by the process of FIG. 32, according to some embodiments.

FIG. 40 is a eighth example graphical user interface that can be provided by the process of FIG. 32, according to some embodiments.

FIG. 41 is a ninth example graphical user interface that can be provided by the process of FIG. 32, according to some embodiments.

FIG. 42 is a tenth example graphical user interface that can be provided by the process of FIG. 32, according to some embodiments.

FIG. 43 is a eleventh example graphical user interface that can be provided by the process of FIG. 32, according to some embodiments.

FIG. 44 is a twelfth example graphical user interface that can be provided by the process of FIG. 32, according to some embodiments.

FIG. 45 is a thirteenth example graphical user interface that can be provided by the process of FIG. 32, according to some embodiments.

FIG. 46 is a fourteenth example graphical user interface that can be provided by the process of FIG. 32, according to some embodiments.

FIG. 47 is a fifteenth example graphical user interface that can be provided by the process of FIG. 32, according to some embodiments.

FIG. 48 is a sixteenth example graphical user interface that can be provided by the process of FIG. 32, according to some embodiments.

FIG. 49 is a seventeenth example graphical user interface that can be provided by the process of FIG. 32, according to some embodiments.

FIG. 50 is a eighteenth example graphical user interface that can be provided by the process of FIG. 32, according to some embodiments.

It should be understood that the drawings are not necessarily drawn to scale. It is to be further appreciated that the following detailed description is merely exemplary in nature and is not intended to limit the invention or the application and uses thereof. Hence, although the present disclosure is, for convenience of explanation, depicted and described as certain illustrative embodiments, it will be appreciated that it can be implemented in various other types of embodiments and in various other systems and environments.

### DETAILED DESCRIPTION

The present disclosure relates to methods and systems for providing a visual representation of material removal from an object by a cutting tool during a cut procedure. For example, the method and system disclosed herein may be used to provide a visual representation of the progress of bone material removal from bone during a surgical bone resection procedure. In other non-limiting examples, the method and system disclosed herein may be used provide a visual representation of material removal in a machining or milling operation. The visual representation may be provided at a display that is updated continuously as the cutting tool penetrates the object so that a user (e.g., a surgeon, etc.) can monitor the progress of material removal in real time (perceived real time). This allows the user to confirm that the planned volume of the object has been removed and obtain a notification if the cutting tool penetrates beyond the planned volume (and a visualization of such penetration). Below is a list of terms and phrases used throughout the present disclosure and their meanings as used herein, many or all of which are apparent to those with ordinary skill in the art.

Constructive solid geometry (CSG): a technique for displaying complex geometrical models by combining multiple models using mathematical operations, such as the Boolean set operations of union, intersection, and subtraction.

CSG operations: the mathematical operations used to combine multiple models to display complex geometrical models, such as the Boolean set operations of union, intersection, and subtraction.

Voxel: volumetric pixel (the volumetric equivalent of a pixel); each of an array of discrete elements into which a representation of a three-dimensional object is divided. Each voxel has a 3D coordinate and holds the color at that coordinate.

Object space: represented in a three-dimensional voxelized grid.

Rasterization: concept in computer graphics describing how a polygonal geometry is converted into a raster (pixel or voxel) image.

A-Buffer rasterization: a visible surface detection technique used to identify what is visible in a scene from a specific viewing point of a virtual camera; an extension of the Z-buffer method which adds transparency. A-buffer rasterization creates an A-buffer containing values describing the depth complexity of the scene.

Fragment: one of a plurality of surfaces making up a pixel and contributing to the color of the pixel.

Cut trajectory: a path that a cutting tool follows during a cut procedure; the volume that the cutting tool has swept through the object up to the current point in time.

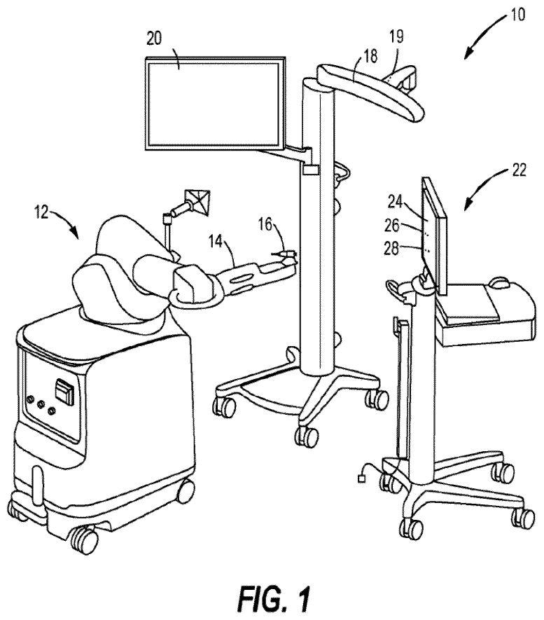

Referring now to the drawings, and with specific reference to FIG. 1, a system 10 for performing a cut procedure on an object and providing a visual representation of material removal from the object during the cut procedure is shown. As a non-limiting example, the cut procedure may be a surgical tissue or bone resection procedure, such as a hip or knee arthroplasty. Alternatively, the cutting procedure may be a machining or milling procedure. The system 10 may include a robot system 12 having a robotic arm 14 that holds and moves a cutting tool 16 (e.g., a burr, saw, reamer, etc.) during the cut procedure. The system 10 may further include a tracking device 18 having an optical localization system 19 (including an optical localization camera) that tracks the poses (positions/orientations) of the cutting tool 16 and the object to be cut (e.g., bone, etc.) during the cutting procedure. The tracking device 18 may be positioned at a stand or at another location separate from the robot system 12, or it may be positioned at or incorporated into the robot system 12. In addition, the system 10 may include one or more display screens 20 for displaying the visual representation of the cut procedure, and a navigation system 22 in communication with the tracking device 18 and the display screen 20. The navigation system 22 may include a computer 24 having a computer processing unit (CPU) 26 configured to perform one or more algorithms that compute and render the visual representation, and output the visual representation to the display screen 20 as described in further detail below. In some arrangements, the CPU 26 may include a graphics processing unit (GPU) 28 to assist the CPU 26 in performing the rendering calculations.

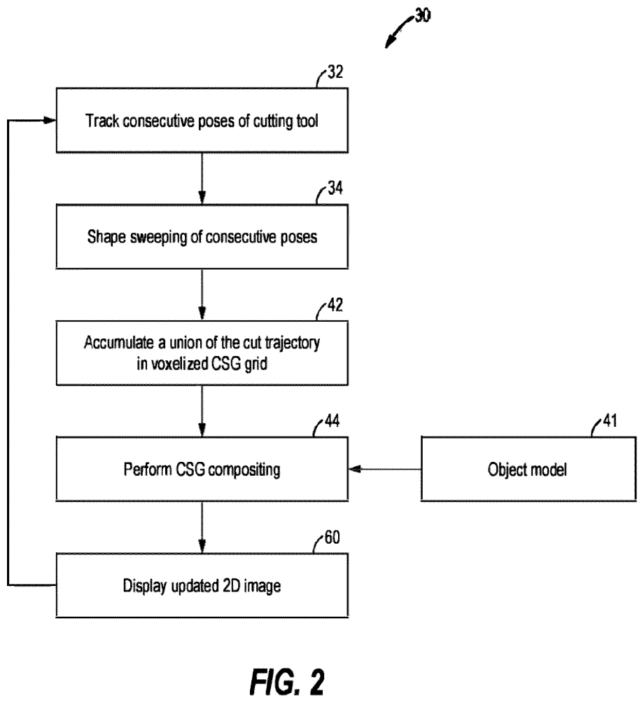

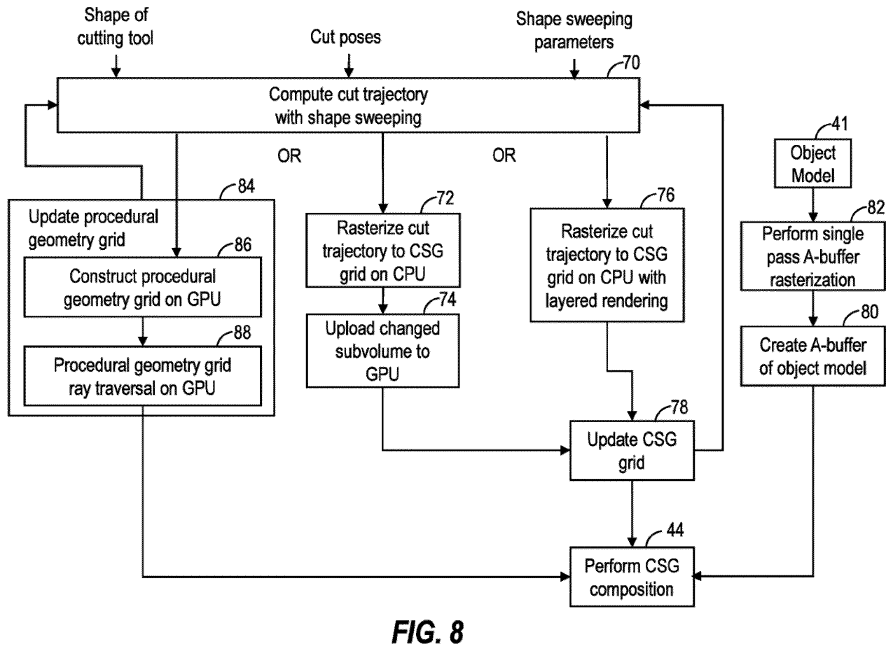

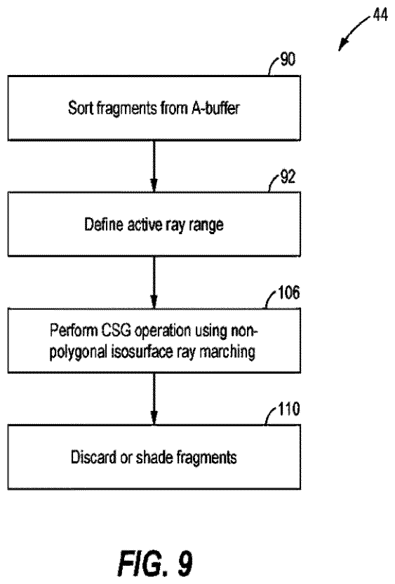

Turning now to FIG. 2, a method 30 for generating the visual representation of the cut procedure as can be performed by the CPU 26 is shown. Further details of the steps of the method 30 are provided below with reference to FIGS. 8-9. At a first block 32, the consecutive poses of the cutting tool 16 as it follows its cutting trajectory during the cut procedure may be tracked based on input from the optical localization system. Each of the consecutive poses represents the discrete position/orientation of the cutting tool 16 at a particular point in time as captured by the optical localization system.

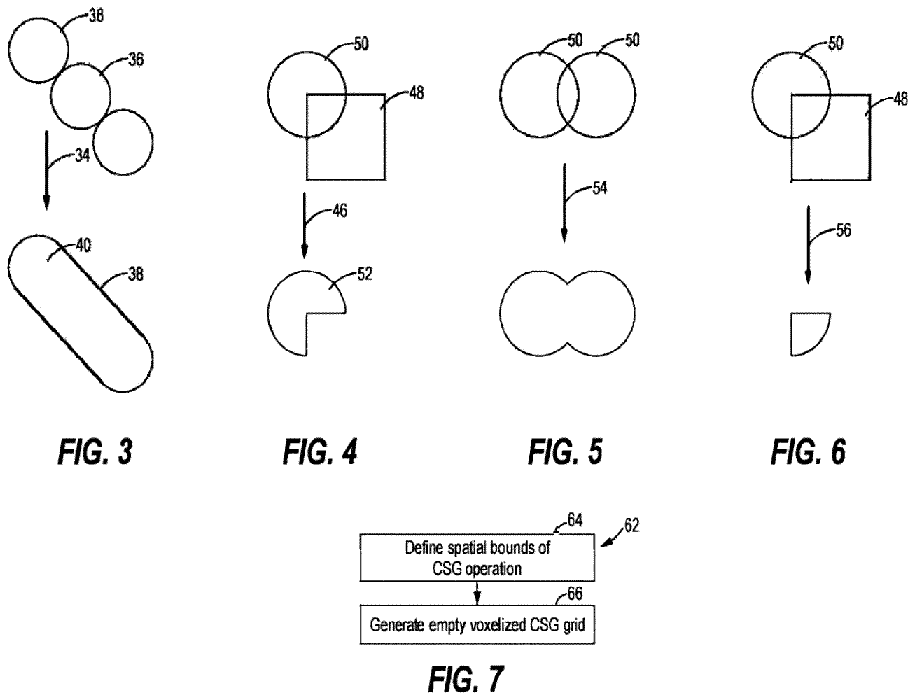

Shape sweeping of the consecutive poses of the cutting tool 16 up to the particular point in time may be performed according to a block 34. The concept of the shape sweeping as performed during the block 34 is schematically depicted in FIG. 3. Although the cutting tool 16 may, in actuality, follow a smooth cutting path/trajectory during the cut procedure, the tracked consecutive poses 36 of the cutting tool 16 may be detected as discrete shapes due to sparse temporal sampling of the poses 36 by the tracking device 18. For instance, if the cutting tool 16 is a burr, the tracked consecutive poses 36 may be distinct spherical shapes, as shown in FIG. 3.

To correct for the temporal resolution of the tracking device 18 and provide a smoother visual output that more closely reflects the smooth cutting path followed by the cutting tool 16, shape sweeping 34 may be applied to the consecutive poses 36, as shown. The shape sweeping 34 may enclose the consecutive poses 36 in a smooth surface 38 to provide a smooth representation of a cut trajectory 40 followed by the cutting tool 16. The shape sweeping procedure provides a smoother visual representation of the cut object as explained further below with reference to FIG. 13.

In the example of FIG. 3, the shape sweeping 34 combines the spherical poses 36 into a capsule-like structure having the smooth surface 38. However, it is noted that shape sweeping as disclosed herein may be applied to a variety of cutting tools having different shapes and may be performed on any closed pose geometry such as, but not limited to, burr-ellipsoid/sphere, saw-rectangular cuboid, and reamer/half-sphere shapes. The swept shape may be generated in any suitable fashion, such as via piecewise linear or higher order (e.g., spline) approximation.

Turning back to FIG. 2, a union of the cut trajectory 40 may be accumulated in a three-dimensional voxelized constructive solid geometry (CSG) grid to produce an object space representation of the union of the cut trajectory 40 up to the particular point in time in the cut procedure (block 42). The voxelized CSG grid may be continuously updated with the union of cut trajectory as the cutting procedure progresses. That is, the voxelized CSG grid may be iteratively updated with the current cut trajectory as the position of the tool changes and removes more material from the object during the cut procedure (see below).

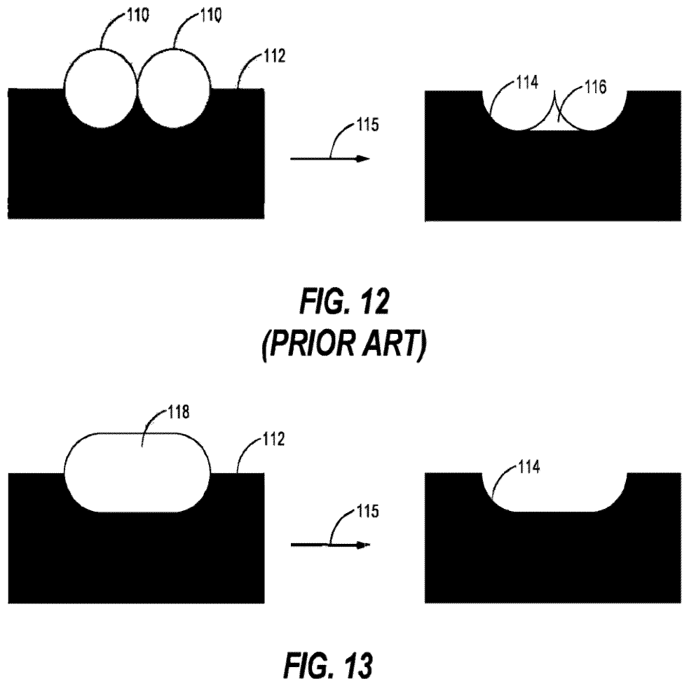

According to a next block 44, CSG compositing may be performed. In addition to other steps described further below with reference to FIG. 9, the CSG compositing step may involve performing a CSG operation on the object space representation of the union of the cut trajectory 40 and a three-dimensional model 41 of the object that is being cut. For example, if the object is bone, the three-dimensional model of the bone may be generated by segmenting a computerized tomography (CT) scan of the bone. If the object being cut is of a softer nature, e.g., flesh or a tumor, the three-dimensional model may change lightly between sequential renderings. The CSG operation may be performed using non-polygonal iso-surface ray marching as described further below with reference to FIG. 12.

In the context of a surgical bone resection procedure, for example, the CSG operation performed during the block 44 may be subtraction of the union of the cut trajectory 40 from the bone model to produce a 2D image of the resected bone. However, depending on the procedure that is performed on the object, other types of CSG operations may be performed such as union, intersection, and painting. Further descriptions of some of the types of CSG operations that may be performed during the block 44 are provided below.

In the CSG operation of subtraction (see FIG. 4), one object is subtracted from a primary object. The operation of subtraction produces a solid having a boundary that is made up of (a) all external points of the primary object that lie outside of the subtracted object, and (b) all external points of the subtracted object that lie inside the primary object. For example, the subtraction 46 of a cube 48 intersecting a quarter of a sphere 50 may be a three-quarter sphere 52 (see FIG. 4). In the CSG operation of union (see FIG. 5), an object is created that combines two or more base objects. Points that are on the boundary of one of the combined objects but are also located internal to other objects are not visible in the combined object. The union 54 of two spheres 50 is schematically depicted in FIG. 5. In the CSG operation of intersection, an object is created from base objects where the boundary is made up of all points that are both external to one object and internal to the other object. For example, FIG. 6 depicts the result of the intersection 56 of a sphere 50 with a cube 48.

Referring again to FIG. 2, upon completion of the block 44, the CPU 26 may output a 2D image of the result of the CSG operation to the display screen 20 (block 60). The 2D image may be continuously updated throughout the cut procedure by repeating the method 30 and continuously displaying the updated 2D image at the display screen 20. The method of FIG. 2 may be repeated with enough frequency so that the 2D image is updated in real time, or at least with enough frequency so that the user perceives the updates as occurring in real time (perceived real time).

Prior to accumulating the cut trajectory 40 in the voxelized CSG grid, an empty voxelized CSG grid may be generated according to the method 62 of FIG. 7. Beginning at a block 64, the spatial bounds of the CSG operation may be defined as the intersection of the bounding box (geometric surface) of the object that is cut and the bounding box of the allowed volume of the object that can be removed during the cut procedure. Optionally, a margin may be added to the intersection to allow cuts made outside of the allowed volume to be visualized at the display screen. Once the spatial bounds of the CSG operation has been defined by the block 64, the empty voxelized CSG grid is generated within the defined spatial bounds according to a block 66.

Turning now to FIG. 8, steps that may be involved in the method 30 of FIG. 2 are schematically depicted. At a block 70, the cut trajectory 40 may be computed using shape sweeping 34 taking into account the shape of the cutting tool 16, the tracked consecutive poses 36, as well as the parameters for shape sweeping (also see FIG. 3). The cut trajectory 40 may then be rasterized into the voxelized CSG grid using the GPU 28 or a combination of the CPU 26 and the GPU 28. For instance, the rasterization may be performed on the CPU 26 according to a block 72, and only the sub-volume of the cut trajectory 40 that has changed since the last update may be transferred to the GPU 28 according to a block 74. Alternatively, the rasterization of the cut trajectory 40 may be performed on the GPU 28 according to a block 76. If performed on the GPU 28, the rasterization may be carried out with layered rendering (layer-by-layer rasterization) since the GPU 28 may only be capable of rendering 2D images. As such, one may decide to perform the rasterization by blocks 72 and 74, or by block 76, depending on which strategy is more efficient. FIG. 8 also shows an option of using a procedural geometry grid (block 84), described below.

Upon completion of rasterization of the cut trajectory 40, the voxelized CSG grid may be updated with the changed sub-volume of the cut trajectory 40 according to a next block 78. By continuously repeating the blocks 70, 72 and 74 (or 76), and 78 throughout the cut procedure, the union of the cut trajectory 40 may be iteratively updated in the voxelized CSG grid up to the current point in time of the cut procedure.

In addition, an A-buffer of the object model 41 may be created (block 80) by performing single-pass A-buffer rasterization on the object model 41 without a depth test (block 82). Single-pass A-buffer rasterization may reduce the cost of the rasterization as more passes increases the cost of the computation. The A-buffer may be a data structure describing the depth complexity of the object in image space from a viewpoint of a virtual camera. This A-buffer and the updated voxelized CSG grid may be used for the CSG compositing step (block 44) as described in further detail below.

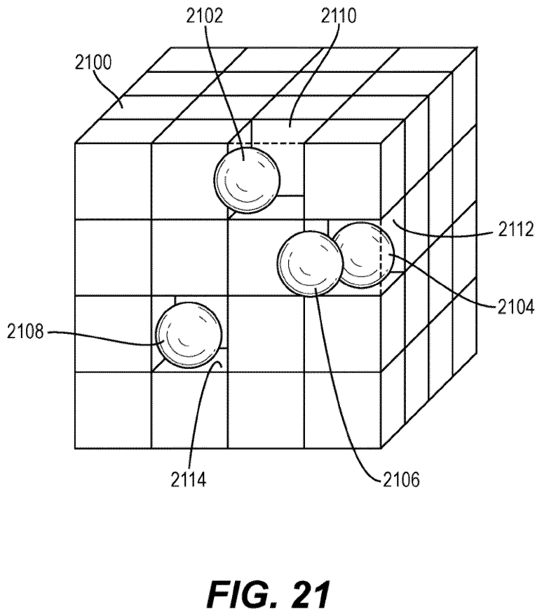

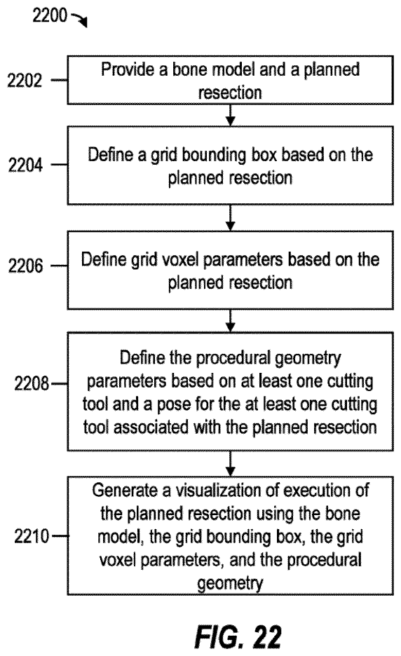

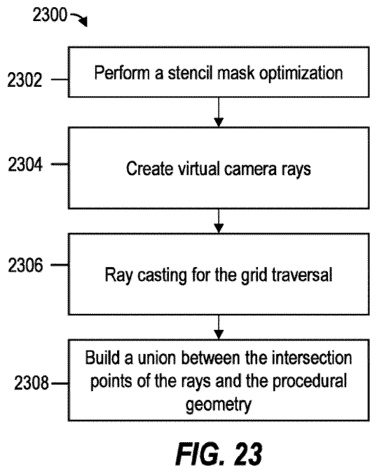

In an embodiments where a procedural geometry grid is used (i.e., block 84 is executed), the cut trajectory accumulated with shape sweeping in block 70 is provided to block 84. Block 84 includes construction of a procedural geometry grid on the GPU 28 at block 86, followed by ray traversal of the procedural geometry grid on the GPU 28. As used herein, procedural geometry refers to a shape that is represented mathematically (e.g., general primitive), and can include, for example, a sphere, a capsule, a cylinder, a box, a plane, a cone, a wedge, etc. For example, a sphere can be represented mathematically by a center point and a radius (e.g., rather than a surface of tessellations or other approach). The procedural geometry grid refers to a three-dimensional bounding box (e.g., axis-aligned bounding box) divided into voxels, where each voxel is associated with a list (e.g., linked list) of procedural geometry. FIG. 21 shows an example illustration of a procedural geometry grid 2100 having associated procedural geometry, shown as a first sphere 2102, a second sphere 2104, third sphere 2106, and fourth sphere 2108. As shown, the first sphere 2102 is associated with a first voxel 2110, while the second sphere 2104 and third sphere 2106 are associated with a second voxel 2112, and the fourth sphere 2108 is associated with a third voxel 2114. Different numbers of different procedural geometries can occupy a voxel in various embodiments. This approach allows the mathematical ideal of such spheres and other shapes to be represented directly in the approach used for visualization in block 84. Further details of the is approach are described in FIGS. 22-23 below. Block 84 is thereby adapted to provide a grid that can be used in the CSG composition step of block 44, according to some embodiments.

Turning now to FIG. 9, steps that may be involved in performing CSG compositing (block 44) are shown. At a first block 90, fragment sorting from the object model A-buffer may be performed. Specifically, the block 90 may involve reading the fragments from the A-buffer and sorting the fragments according to their depths.

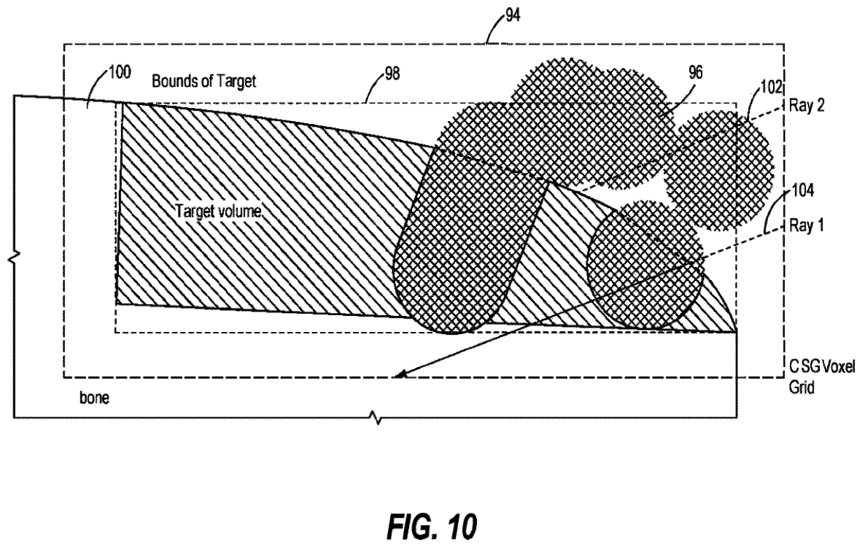

At a block 92, the active ray range for non-polygonal iso-surface ray marching may be defined to reduce the active ray range for the ray marching and increase the efficiency of the computation. A schematic illustration of defining the active ray range as performed during the block 92 is shown in FIG. 10. FIG. 10 shows the voxelized CSG grid 94, the union of the cut trajectory 96, and a bounding box 98 of the allowed volume of the object 100 (e.g., bone) that is allowed to be cut during the cut procedure. In non-polygonal iso-surface ray marching, a plurality of rays (e.g., rays 102 and 104) from a particular viewpoint of a virtual camera may be cast or marched through the voxelized CSG grid 94 to iteratively search for intersection points. The intersection points may be used to produce an image space (2D) surface representation of the cut procedure that is displayed at the display screen 20. Each of the rays 102 and 104 may correspond to a pixel in the 2D image that is produced. Defining the active ray range according to the block 92 restricts the search for ray marching and increases the efficiency of the CSG operation. More particularly, according to the block 92, ray marching may be skipped (or “inactive”) if the bounding box 98 of the object 100 and the voxelized CSG grid 94 do not overlap along the ray (see dashed sections of exemplary rays 102 and 104 corresponding to the “inactive” ray range). In the “inactive” ray range, no computation is performed. Otherwise, the rays are iterated in the “active” ray range as shown by the solid section of the ray 104. In the “active” ray range, the start of the marching is either the closest surface of the object 100 (from the A-buffer) or where the ray enters the voxelized CSG grid 94, whichever is farther (equation 1). The ray marching is terminated at the furthest intersection of the ray and the object 100 (from the A-buffer), or if CSG grid 94 is exited, whichever happens earlier (equation 2). For example, in FIG. 10, the dashed sections of the exemplary rays 102 and 104 are skipped during marching because they lie outside of the object 100, with ray 102 being terminated as soon as the object 100 is reached since at that point the CSG grid 94 is not set.

$${t}_{start} = max ( {t}_{object \_ closest} , {t}_{grid \_ entry} ) \qquad ( equation 1 )$$

$${t}_{end} = min ( {t}_{grid \_ exit} {t}_{object \_ furthest} ) \qquad ( equation 2 )$$

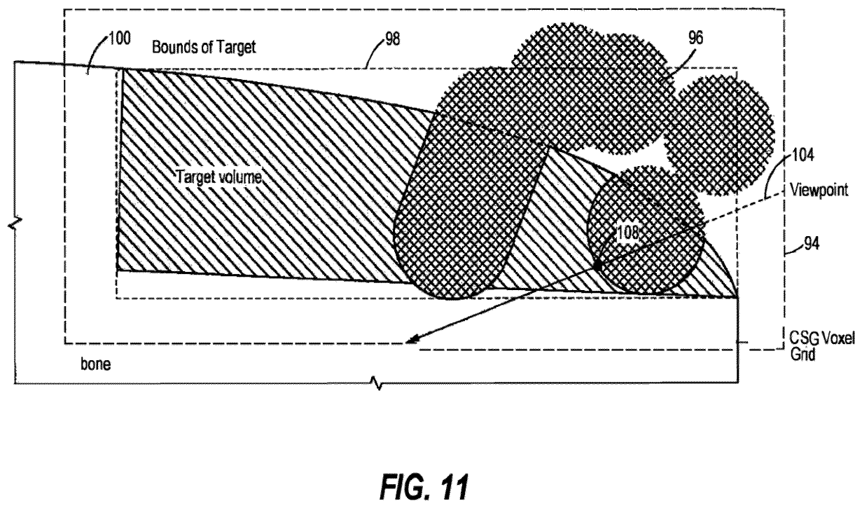

With the active ray range defined as described above, the CSG operation may be performed on the object 100 (from A-buffer) and the union of the cut trajectory 96 using non-polygonal iso-surface ray marching (block 106). It will be appreciated that other CSG operations may be implemented, e.g., surface painting, intersection, etc. FIG. 11 depicts an example of using non-polygonal iso-surface ray marching to display the results of subtracting the union of the cut trajectory 96 from the object 100. For each pixel, a ray 104 is marched through the active ray range (solid portion of the ray 104) from a viewpoint of the virtual camera, and the intersection point 108 is determined as the closest intersection point that is inside the object 100 and outside of the union of the cut trajectory 96. The intersection point 108 represents the cut surface of the object 100, and is used to produce the 2D surface representation that is displayed at the display screen 20.

If the object 100 and the union of the cut trajectory 96 do not overlap along a ray (indicating that the object has not been cut at that location), the object surface is displayed as uncut in the 2D image. Iteratively searching the voxelized CSG grid 94 for intersection points in this manner produces the 2D surface representation of the object as it is cut during the cut procedure. For example, in the context of bone resection, the block 106 may result in a 2D surface representation of the resected bone surface.

Referring again to FIG. 9, the CSG compositing block 44 may further include discarding or shading fragments of the 2D surface representation produced from the CSG operation to refine the 2D image (block 110). In particular, during the block 110, a machining calculation may be applied that determines the shading of each fragment and determines the red, green, and blue color contribution of the fragments at each pixel. In the context of surgical bone resection procedures, regions of bone that are to be resected may be colored in green, and regions where the cutting tool 16 has penetrated beyond the planned region may be colored in red. The block 110 may further involve discarding the fragments that are not visible. The refined 2D image may be displayed at the display screen 20 (block 60, FIG. 2).

In a further embodiment, a display of the total removed material, total material remaining to be removed, or both may be generated for the user's guidance. For example, the display may indicate that 80% of the material to be removed has been removed, and 20% of the material to be removed remains on the object.

The present application applies shape sweeping 34 to produce a smoother visual representation of the cut procedure. FIG. 12 shows an object surface 114 produced after subtracting un-swept cut poses 110 from an object model 112 in accordance with the prior art. As can be seen, due to the temporal resolution of the tracking method used to track the poses of the cutting tool 16, the poses 110 may be detected as discrete shapes representing the shape of the cutting tool 16. As a result, the displayed object surface 114 after subtraction 115 of the un-swept cut poses 110 may have edgy, wave-like ridges 116 as artifacts of the sparse temporal sampling of the cut poses 110. In contrast, the subtraction of swept cut poses 118 from the object model 112 in accordance with the present disclosure avoids the leftover ridges and produces a smoother visual representation of the cut object surface 114.

In general, it can therefore be seen that the technology disclosed herein may find applicability in a variety of settings such as, but not limited to, surgery and machining. More specifically, the present disclosure may be applicable in various applications that may benefit from real time visualization of the progress of material removal from an object during a manual or automated cut procedure. Although robotic bone resection is given as one example, the technology disclosed herein may also be applicable to manual surgery, such as manual bone resection, to cutting or milling operations, as well as other types of operations.

Current methods used to produce a visual representation of bone resection procedures may be computationally expensive due to the large number of tool positions that need to be subtracted from the bone mod The computational cost of current methods that rely on depth peeling may increase with increasing depth complexity of the scene. Moreover, the results of the computation may be invalidated and require re-computation if the virtual camera is moved or rotated.

By contrast, the approach discussed above has a constant computational cost with respect to the depth complexity of the scene. The computation of the union of the cut trajectory as disclosed herein happens incrementally at a constant computational cost by rasterizing the model of the cut trajectory to a voxelized CSG grid that (spatially) covers the planned resection volume (with an optional margin). Creating the image-space fragments that describe the voxelized model in a pixel also happens at the constant computational cost of marching rays through the voxelized CSG grid. The resolution of the voxelized CSG grid determines the computational cost, and is fixed beforehand. The voxelized CSG representation also removes the burden of processing overlapping or duplicate positions. Furthermore, high visual quality of resected surfaces is achieved by sub-voxel accurate filtering during non-polygonal iso-surface ray marching. The initial iso-surface intersections are refined in a binary search manner by reducing the marching step size.

As disclosed herein, the voxelized CSG representation may be rasterized on the CPU or on the GPU by layered rendering of the CSG grid into slices, depending on which rasterization strategy is more efficient. If the rasterization happens on the CPU, only the sub-volume of the cut trajectory that has changed since the last update is uploaded to the GPU to further enhance efficiency. This sub-volume is defined by the bounding box of the updated cut pose of the cut trajectory.

The present disclosure also offers another improvement to the quality of the visual representation over existing methods. In current technologies, the consecutive poses of the cutting tool may be too far apart due to sparse temporal sampling of the tool position tracking device, resulting in leftover ridges in the displayed image that may not accurately reflect the smooth cutting path followed by the cutting tool. The present disclosure applies shape sweeping to represent the consecutive poses within a smooth enclosing surface to prevent ridge artifacts from appearing in the visual representation of the cut procedure. Advantageously, this results in visually smoother resected surfaces in the displayed image which may more closely represent the true appearance of the resected object.

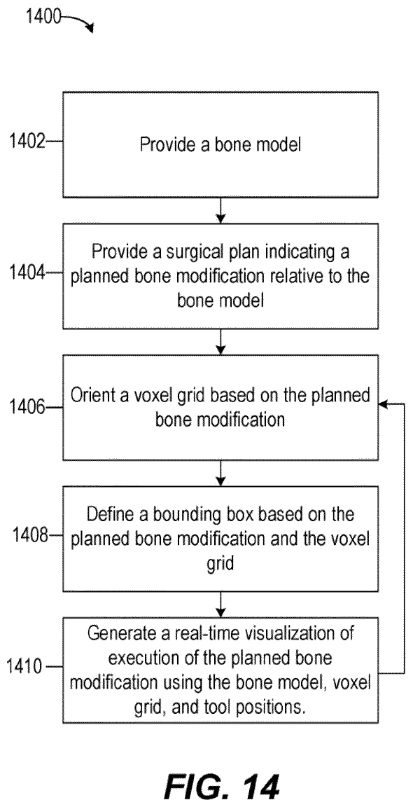

Further technical advantages can be achieved by additionally or alternatively applying the features described below with reference to FIGS. 14-19. As described in detail below, computational expense can be further reduced by orienting a graphics coordinate system (e.g., voxel grid, voxelized CSG grid 94) and/or bounding a volume for visualization calculations (e.g., defining bounding box 98) based on a cutting plan (e.g., surgical plan), for example by reducing the total volume for rasterization and CSG operations while capturing the areas of interest for the user. Such an approach can also reduce visualization artifacts, for example by aligning a voxel grid (e.g., voxelized CSG grid 94) with the planned cut such that successfully performing the planned cut results in a surface which is inherently smooth in the aligned voxel gird. Thus, as will be apparent from the following description, technical improvements in graphical visualization technologies specifically adapted for the technical field of surgical navigation and guidance and analogous applications are provided by the features shown in 14-19 and detailed below.

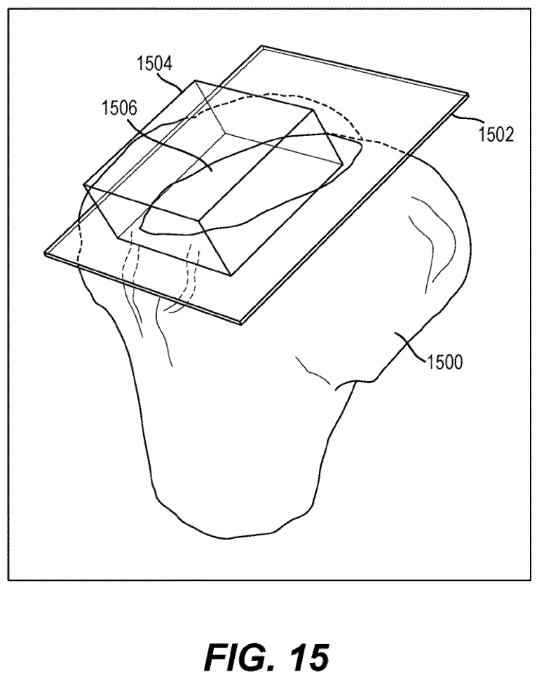

Referring now to FIG. 14, a flowchart of a process 1400 is shown, according to some embodiments. Process 1400 can be executed by surgical system, for example a robotic surgery system, surgical navigation system, etc. in various embodiments. For example, in some embodiments, process 1400 can be executed by the system 10, for example by or using the navigation system 22 (e.g., by the CPU 26). The process 1400 can optionally be executed in combination with method 30 and/or method 62 described above, in some embodiments. The discussion of FIG. 14 refers to FIG. 15 for sake of example, which shows an illustration of a bone model 1500, planned bone modification plane 1502, and bounding box 1504, according to some embodiments.

At step 1402, a bone model is provided. The bone model is a three-dimensional virtual representation of a bone. For example, in some embodiments the bone model is generated from segmented CT images (or other types of medical imaging) of a patient's bone. In some embodiments, the bone model is a generic (standard, non-patient-specific) model of a bone. As one example, FIG. 15 shows a bone model 1500 of femur.

At step 1404, a surgical plan is provided which indicates a plan (planned cut, cut plan, planned bone modification) for a bone modification (cut, burr, drill, sculpt, etc.) relative to the bone model. The surgical plan can also indicate one or more additional bone modifications (additional planned cuts), for example a sequence of bone modifications planned to prepare a surface of a bone to receive an implant. In some embodiments, step 1404 includes automatically generating the surgical plan based on patient characteristics and/or providing a graphical user interface that allows the user to adjust the surgical plan (e.g., by adjust a planned implant position relative to the bone model). In some examples, the planned cut indicated by the surgical plan is a plane (or is planar) and is defined relative to the bone model. For example, a plane having an orientation and position relative to a position and orientation of the bone model can be provided in step 1404. As one example, FIG. 15 shows a planned bone modification plane 1502 positioned and oriented relative to the bone model 1500.

At step 1406, a voxel grid (e.g., voxelized CSG grid 94) or other graphics coordinate system is oriented relative to the bone model based on the planned bone modification. Orienting a graphics coordinate system can be performed in step 1406 such that the planned bone modification has a constant value in one dimension of a three-dimensional graphics coordinate system. For example, where the planned bone modification is planar, step 1406 can include aligning the voxel grid to the planed bone modification such that a first dimension of the voxel grid is perpendicular to the planned bone modification, a second dimension of the voxel grid is parallel to the planned bone modification, and a third dimension of the voxel grid is parallel to the planned bone modification. In the example of FIG. 15, step 1406 includes orienting the voxel grid based on the planned bone modification plane 1502 such that the voxel grid (which itself has orthogonal dimensions, e.g., cubical voxels in the example of FIG. 15) has two coordinate dimensions parallel to the planned bone modification plane 1502 and one coordinate dimension perpendicular to the planned bone modification plane 1502, i.e., such that the planned bone modification plane 1502 is constantly-valued in one dimension of the voxel grid and extends in all directions in the other two dimensions of the voxel grid.

Orienting the voxel grid or other graphics coordinate system relative to the bone model in step 1406 can also be characterized as orienting the bone model relative to the voxel grid. Coordinate transformations can be used to translate between the voxel grid aligned to the planned bone modification and other coordinate systems used in a navigation or control process (e.g., used by system 10) (e.g., used by a tracking system, used for robotic control, etc.).

At step 1408, a bounding box is defined based on the planned bone modification and the voxel grid (or other graphics coordinate system). The bounding box can be a cube or rectangular box defined square to the voxel grid (e.g., with each surface of the bounding box being planar and having a constant value in a dimension of the graphics coordinate system), which can simplify calculations as compared to defining the bounding box in some other manner. Step 1408 can include spatially positioning the bounding box with one surface at or slightly to a first side of the planned bone modification (e.g., by one millimeter, two millimeters, three millimeters, etc.) with a remainder of bounding box to a second, opposing side of the planned bone modification. Step 1408 can also include sizing the bounding box such that all portions of the bone model on the second side of the planned bone modification (i.e., the portion(s) planned for resection) are enclosed in the bounding box, and, in some embodiments, limiting the extent of the bounding box to the extremities of such portions of the bone model in each coordinate dimension (e.g., with a slight offset).

FIG. 15 illustrates an example where the bounding box 1504 is positioned at the planned bone modification plane 1502 and sized to enclose a portion 1506 of the bone model 1500 planned for resection (i.e., portions separated from the remainder of the bone model 1500 by the planned bone modification plane 1502. The bounding box 1504 is thereby narrowly scoped to include the portions of the bone model 1500 intended to be affected during execution of the surgical plan to make the planned bone modification without encompassing substantial portions of the bone model not intended to be affected by the planned bone modification. Volume of the bounding box 1504 can thus be minimized while capturing all areas of interest to a user for visualization. The bounding box 1504 defined in step 1408 based on the planned bone modification and voxel grid is thereby limited to minimize the amount of rasterization, CSG calculations, ray casting, and/or other calculations required for generating visualizations, thus reducing computational expense and improving computational efficiency.

At step 1410, a real-time visualization of execution of the planned bone modification is generated using the bone model, the voxel grid, and, in some embodiments, data (e.g., from a tracking system) indicative of positions of a tool used to make the planned bone modification. In some embodiments, step 1410 includes determining voxels of the voxel grid shared by bone model and by an accumulation (e.g., sweep) of the tool positions (e.g., using a CSG operation as described above), and updating the visualization to remove (or color, shade, make transparent, etc.) the visualization of such voxels. Step 1410 can include generating a representation of a surface of the bone model resulting from removal of virtual bone from the voxels shared by the bone model and the accumulation of the tool positions. Step 1410 can be executed using the approaches described above with reference to FIGS. 1-13, for example.

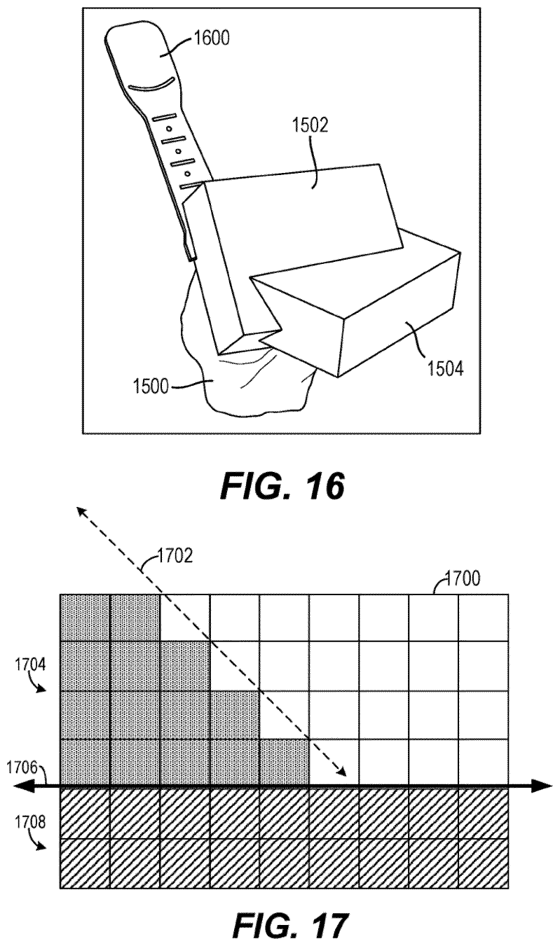

Due to alignment of the planned bone modification with the voxel grid (or other graphics coordinate system), the visualizations generated in step 1410 in inherently-smooth surfaces when the bone modification is executed according to plan. FIGS. 16-17 include schematic diagrams illustrative of such effects. FIG. 16 illustrates a cutting tool 1600 relative to a bone model 1500 along with a plan-aligned box 1602 and a bone-axis-aligned box 1604. The plan-aligned box 1602 illustrates the orientation of a voxel grid arranged relative to the bone model 1500 according to the steps of process 1400, while the bone-axis-aligned box 1604 illustrates a voxel grid aligned to the bone model 1500 agnostic to the planned bone modification. As shown in FIG. 16, a surgeon following the surgical plan will operate the cutting tool 1600 along a direction parallel to a surface of the plan-aligned box 1602 (e.g., along a coordinate direction of the voxel grid), i.e., the cutting tool 1600 aligned to the plan and thus the box 1602 as intended. Such alignment and parallel movements can simplify calculations and reduce visualization artifacts. In contrast, the surgical tool 1600 is shown at an angle relative to all surfaces and coordinate directions of the bone-axis-aligned box 1604, which can lead to visualization artifacts such as steps between voxels as shown in FIG. 17.

FIG. 17 illustrates a grid 1700 (shown in two dimensions for the sake of explanation, with the principles illustrated extendable to three or more dimensions). As shown in FIG. 17, a first line 1702 extends across the grid 1700 in a direction non-parallel to either (any) coordinate dimension of the grid 1700. FIG. 17 also shows the boxes 1704 (voxels) of the grid 1700 beneath the first line 1702 as shaded/dotted. Because the first line 1702 is at an angle relative to the grid 1700, the boxes 1704 beneath the first line 1702 form steps (ridges, jumps, artifacts, etc.). A visualization generated using the boxes 1704 will thus include visualization artifacts, i.e., the steps, thus inaccurately representing the straight line 1702.

FIG. 17 also shows a second line 1706 aligned with the grid 1700. The second line 1706 can be aligned with the grid 1700 as result of orienting the grid 1700 based on a surgical plan as in process 1400. Because the second line 1704 is aligned with the grid 1700, boxes 1708 under the second line 1706 can be highlighted without steps, jumps, discontinuities, etc. A visualization generated of the boxes 1708 thus accurately represents the smooth, linear contour of the second line 1706 without visualization artifacts. In the context of process 1400, line 1704 can correspond to the positions of a tool as the planned bone modification is executed according to the surgical plan. Visualization based on tracking data as the tool moves along such a line (surface, etc.) can thus inherently result in no or limited visualization artifacts, as an effect of aligning the voxel grid based on the surgical plan in step 1406.

In some such embodiments, no or limited post-processing (e.g., smoothing, interpolation, etc.) is required to achieve a smooth appearance of the visualization, such that the visualization more closely corresponds to underlying data (e.g., tracking data) as compared to an approach where some post-processing approach is applied to hide the steps between boxes 1704. Computation efficiency can also be improved by minimizing the need for such post-processing.

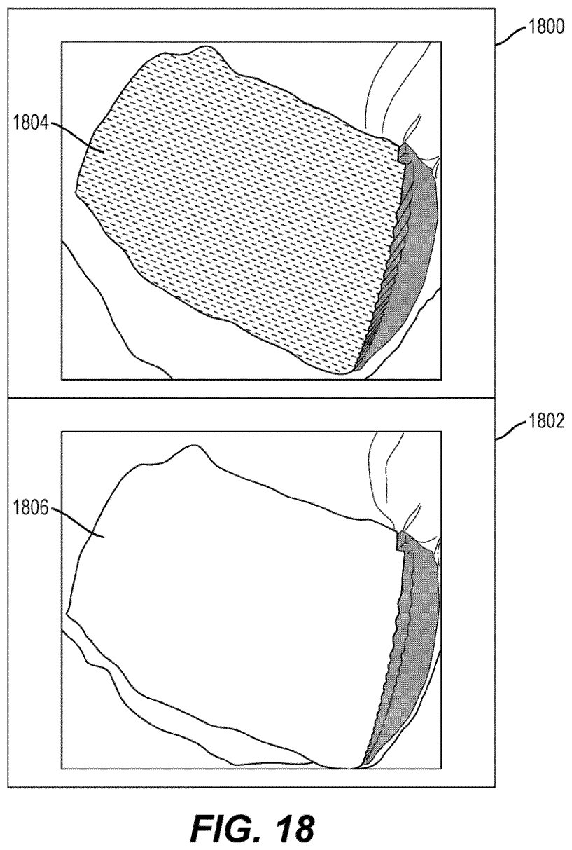

FIG. 18 shows example visualizations, according to experimental results. In particular, FIGS. 18 includes a first illustration 1800 generated without alignment of the voxel grid to the planned bone modification and a second illustration 1802 generated following process 1400 (e.g., including alignment of the voxel grid to the planned bone modification). The first illustrations 1800 includes visualization artifacts caused by misalignment to the voxel grid causing the appearance of a rough surface 1804. The second illustration 1802 is free of such visualization artifacts due to alignment of the voxel grid to a smooth surface 1806 resulting from a bone modification. FIG. 18 thereby provides experimental results showing improvements in visualization quality resulting from the techniques described herein, including by execution of process 1400.

Referring again FIG. 14, FIG. 14 illustrates that the process 1400 can return from step 1410 to step 1406. In particular, for procedures involving multiple planned bone modifications (e.g., multiple cuts at different angles to prepare a femur to receive an implant), process 1400 can include re-orienting the voxel grid based on an additional (subsequent, second, etc.) planned bone modification in response to an indication that a first (preceding, etc.) planned bone modification is completed (e.g., based on a user input, based on detecting from tracking data that a tool completed a full extent of the planned cut, etc.). Re-orienting the voxel can include changing the voxel grid from being aligned with the first planned bone modification to being aligned with the additional planned bone modification (e.g., so that a first dimension of the voxel grid becomes perpendicular to the additional planned bone modification while second and third dimensions of the voxel grid are parallel to the additional planned bone modification). The voxel grid is thus updated (re-oriented, moved, changed, rotated, etc.) for different stages of a surgical procedure (e.g., different cuts, different bone modifications, different subparts of a continuous bone modification, etc.) in order to provide efficient, artifact-free visualizations for multiple stages of a surgical procedure.

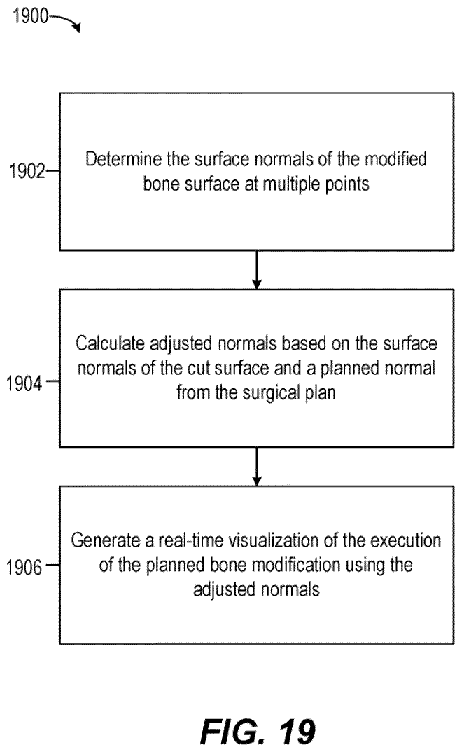

Referring now to FIG. 19, a flowchart of a process 1900 providing a plan-dependent visualization feature is shown, according to some embodiments. Process 1900 can be used with process 1400 in some embodiments, for example as part of generating a real-time visualization of execution of the planned bone modification using the bone model and tool positions as in step 1410 of FIG. 14. Process 1900 may also be used in an implementation which omits some or all of process 1400. Process 1900 can be executed by execution by one or more processors of instructions stored in non-transitory computer-readable media, e.g., by the system 10 for example by or using the navigation system 22 (e.g., by the CPU 26). Process 1900 can advantageously use the surgical plan to eliminate visualization artifacts from the bone surface.

At step 1902, the surface normal of the modified bone surface is determined at multiple points. The multiple points may be points for which the intersection point 108 is within a threshold vicinity of the planned bone modification (e.g., planned cut plane). For example, the points used in step 1902 may be selected as the points for which μnₚₗₐₙ(p−oₚₗₐₙ)∥<ϵ, where p is the location of the intersection point 108, nₚₗₐₙ is the normal vector of the planned bone modification (e.g., planned resection plane), and oₚₗₐₙ is the origin point of the planned bone modification (e.g., planned resection plane), and ϵ is a fixed threshold value, for example selected based on an expected amount of deviation in a cut, tolerance in a cutting workflow, etc. . . . For such points, a normal of the surface of bone modification (e.g., actual resection surface) can be determined, for example by using a volume gradient approach applied to the voxel representation of the bone remaining after the CSG operations described herein. The normal of the surface of the bone modification calculated from the voxel representation for a given point is denoted here as n_{grid}.

At step 1904, adjusted normals are calculated based on nₚₗₐₙ (the normal vector of the planned bone modification) and n_{grid} (the normal of the modified bone surface as calculated from the voxel grid representation). The adjustment may be an interpolation between the two normal. In some embodiments, an adjusted normal n_{adjusted} is calculated as n_{adjusted}=(n_{grid}*α)+(nₚₗₐₙ*(1−α)), where α is weighting parameter. In some embodiments, α has a value between 0.7 and 0.9, for example approximately 0.85. In some embodiments, α is a fixed value. In some embodiments, α is a function of the distance from the intersection point 108 to the planned bone modification.

To apply the normal modification when the resection involves multiple resection planes, some embodiments of process 1900 including keeping track of the associated resection plane during ray-casting of the raster volume. Since each raster volume may have a unique resection plane associated with it, information identifying the corresponding planned surface can be stored with (added to, tagged onto, etc.) intersection point 108. During the CSG subtraction step as described above, this information isused in some embodiments to identify which nₚₗₐₙ and oₚₗₐₙ are used in instances of steps 1902 and 1904.

At step 1906, a real-time visualization of the execution of the planned bone modification is generated using the adjusted normals (i.e., using n_{adjusted} for different points). The adjusted normal can be used in surface shading approach, for example using Phong shading to generate the visualization. Use of the adjusted normals in step 1906 can reduce or eliminate artifacts which appear on the modified bone surface, such that the surface appears flat, smooth, etc. as displayed to a user.

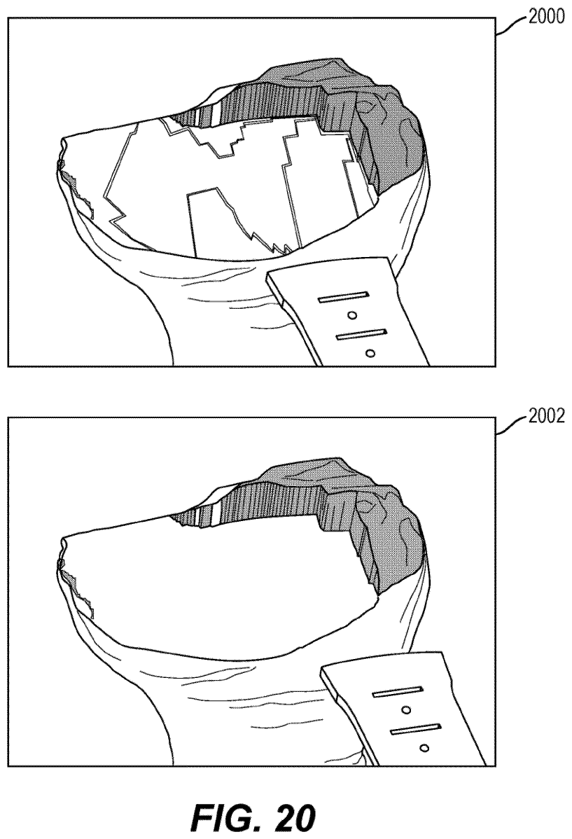

FIG. 20 shows a comparison of a first visualization 2000 prepared without process 1900 and a second visualization 2002 prepared by applying process 1900. In the first visualization 2000, several steps are shown as a result of the voxel representation (steps between levels of the voxel grid). Advantageously, the second visualization 2002 prepared by applying process 1900 shows a smooth modified bone surface without such artifacts. This allows display of a representation of the cut bone surface with a smooth surface as may be expected by the user (e.g., surgeon) to result from the bone modification. Plan-dependent visualization techniques as described herein thereby provide technical solutions in the field of graphical visualization techniques for computer-assisted surgical operations, including with respect to robotic surgery systems.

Referring now to FIG. 22, a flowchart of a process 2200 for providing a visualization using procedural geometry is shown, according to some embodiments. The process 2200 can be executed by the can be executed by execution by one or more processors of instructions stored in non-transitory computer-readable media, e.g., by the system 10 for example by or using the navigation system 22 (e.g., by the GPU 28).

At step 2202, a bone model and a planned resection are provided. The bone model can be provided as described above, for example based on segmentation of pre-operative images (e.g., MRI, CT, etc.) of a patients bone, based on a library of stored bone models, based on interoperative tracking of a probe contacting surfaces of the bone, etc. The planned resection can be provided based on results of a surgical planning workflow, for example provided via a graphical user interface of the system 10. The planned resection can be a surface (E.g., curved surface, plane) and/or volume having a planned pose (location and orientation) relative to the bone model. Data relating to the planned resection provided in step 2202 can include selection of a tool to be used in the planned resection, planned poses for the tool to be in while executing the planned resection, and/or other information relating to the tool to be used in the planned resection.

At step 2204, a grid bounding box is defined based on the planned resection. Step 2204 can include generating a grid, for example divided into axis-aligned bounding box voxels (e.g., in three-dimensions) based on the planned resection. The grid can be sized to encompass the planned resection (e.g., with a surrounding region of interest), for example provided as the minimum size suitable for including the planned resection and a surrounding region of interest. In some embodiments, the grid is oriented based on the planned resection, for example as described above with reference to FIG. 14. Step 2204 can include providing each voxel with a linked list to which procedural geometry data can be added.

At step 2206, grid voxel parameters are defined based on the planned resection. For example, voxel parameters such as voxel size, etc. can be determined as a function of the planned resection, for example so as to provide sufficient accuracy, resolution, etc. while improving computational efficiency, based on the shape of the planned resection.

At step 2208, procedural geometry parameters are defined based on at least one cutting tool and a pose for the at least one cutting tool associated with the planned resection. Data relating to the planned resection provided in step 2202 can include information on the tool to be used, which can be associated in step 2208 with a particular procedural geometry. For example, use of a burr of a particular size can be planned for use in the planned resection, and step 2208 can include defining procedural geometry to include a sphere having a radius of the burr indicated for the planned resection. As another example, use of a router-cutting tool can be indicated for the planned resection, in which scenario step 2208 can include defining procedural geometry to include a half-capsule shape of a size corresponding to the identified router-cutting tool, with a planned orientation of the tool used to orient the half-capsule shape in the procedural geometry. Various such examples are possible as appropriate to represent different tools, different sizes of tools, different orientations of tools, etc. that may be used. The procedural geometry parameters can thus be dynamically assigned based on the planned resection to best represent the particular tool(s) selected for use in a particular planned resection.

At step 2210, a visualization of execution of the planned resection is generated using the bone model, the grid bounding box, the grid voxel parameters, and the procedural geometry. Step 2210 can include a ray-casting and/or CSG operation, for example as shown in FIG. 23 and described in detail with reference thereto below. Step 2210 can include providing surgical guidance and navigation by updating the visualization as the planned resection is executed, based on tracking of the tool, such that the visualization shows actual execution of the resection relative to the planned resection and the bone model. For example, positions taken by the tool during execution of the resection can be represented in the procedural geometry. Advantageously, the visualization is adapted to details of the planned resection, including the tool to be used in executing the planned resection, such that the visualization can accurately represent the planned resection (and execution of the planned resection based on tracking data) in a manner that smoothly represents cuts executed by the tool without artifacts that might otherwise be left by a rasterization approach. Accordingly, the process 2200 may be particularly well-adapted to providing visualizations for curved tools (e.g., spherical burrs) and/or curved resections, for example for guidance of surgical operations on shoulders or hips.

Process 2200 can be executed for different planned resections, for example such that the procedural geometry parameters are updated in accordance with the different planned resections. For example, a first planned resection can be associated with first procedural geometry parameters and guidance for the first planned resection can be provided using the first procedural geometry parameters. A surgical operation can then also include a second planned resection associated with second procedural geometry parameters (e.g., due to use of a second tool, based on a different orientation of the tool when executing the second resection relative to the first resection, etc.), such that the procedural geometry parameters can be updated from the first procedural geometry parameters to the second procedural geometry parameters for use in providing guidance for the second planned resection. Parameters used in visualization are therefore plan-dependent, i.e., variable based on planned resections of a surgical plan.

Referring now to FIG. 23, a process 2300 for providing a visualization using procedural geometry is shown, according to some embodiments. The process 2300 can be executed by the can be executed by execution by one or more processors of instructions stored in non-transitory computer-readable media, e.g., by the system 10 for example by or using the navigation system 22 (e.g., by the GPU 28). Process 2300 can be executed as part of step 2210 of process 2200, in some embodiments.

At step 2302, a stencil mask optimization is performed. The stencil mask optimization can include determining area for ray casting, for example an area where the bone model overlaps the planned resection. Step 2302 can include proving the stencil mask (stencil buffer) to regulate the number of rays that will be cast, for example according to the solution to an optimization problem that minimizes the number of rays or optimizes some other objective to improve computational efficiency of process 2300.

At step 2304, a virtual camera rays are created. The virtual camera rays extend from the point of view of a virtual camera viewing the bone model and planned resection, for example the viewpoint of a virtual camera as mentioned above with reference to FIG. 11. Step 2304 can include creating sufficient virtual rays to view the area defined by the stencil mask from step 2302, for example with each ray associated with a pixel of a display screen for the visualization. Each ray may have a direction defined by the corresponding pixel's coordinate in screen space, and is oriented to intersect a procedural geometry grid (e.g., from process 2200).

At step 2306, ray casting is performed. The rays created in step 2304 are cast and traverse the procedural geometry grid (e.g., the grid generated according to process 220). Various approaches for ray casting grid traversal can be implemented. Ray casting can determine the points at which the procedural geometry (e.g., surfaces thereof) intersect the rays.

At step 2308, a union between the intersection points of the rays and the procedural geometry is built. Step 2308 can include determining area associated with intersections of the rays with the procedural geometry. For example, where the procedural geometry represents tracked positions of a tool during execution of a resection, the union between the intersection points of the rays and the procedural geometry can represent the resection made by the tool. The union can then be used for selecting the colors of pixels associated with various rays. In some embodiments, the output of step 88 can then be provided to block 44 of FIG. 8 for CSG composition or other steps for providing a final visualization via a display screen. Accordingly, the procedural geometry (e.g., defined in a plan-dependent manner in process 2300) can be used in a ray-casting approach to facilitate visualization of surgical operations.

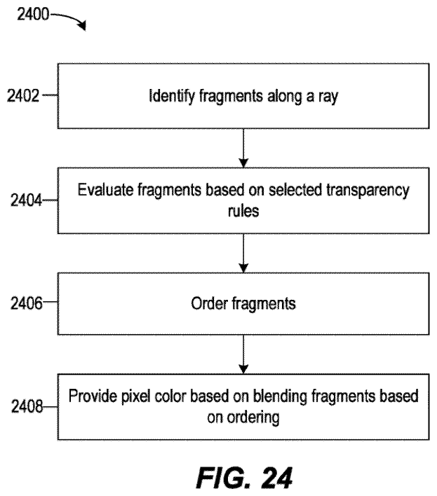

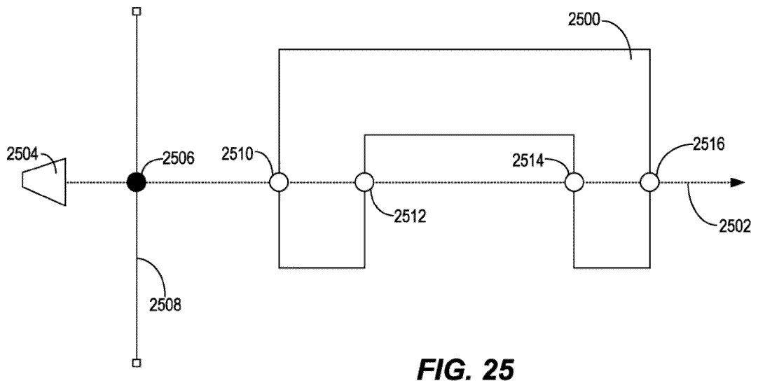

Referring now to FIGS. 24-25, teachings relating to providing transparency of visualizations for surgical planning and navigation are shown, according to some embodiments. FIG. 24 illustrates a process 2400 and FIG. 25 provides an example illustration to facilitate understating of process 2400. The process 2400 can be executed by one or more processors of instructions stored in non-transitory computer-readable media, e.g., by the system 10 for example by or using the navigation system 22 (e.g., by the GPU 28). The process 2400 can be executed in combination with any of the various other processes and approaches described herein, for example when transparency of one or more surfaces or objects represented in the visualizations herein is desired (e.g. transparency of a bone model, transparency of an implant, transparency of a cutting tool, transparency of a planned resection, combinations of the foregoing, etc.).

At step 2400, fragments are identified along a ray. FIG. 25 illustrates an example of identification of fragments of an object 2500 along a ray 2502. The ray 2502 is cast from a virtual camera 2504 and is associated with a pixel 2506 at plane 2508. Due to the geometry of the object 2500, the ray 2502 crosses a border of the object 2500 four times, with the crossing points identified as fragments. In particular, as shown in FIG. 25, process 2400 would identify four fragments: first fragment 2510, second fragment 2512, third fragment 2514, and fourth fragment 2516. Various other geometries will result in other fragments being identified, with a ray—in most scenarios—identifying a fragment at least at a front surface of an object and a back surface of the object, potentially with intermediate fragments for hollow objects, objects intersected by other objects, etc.

At step 2404, the fragments are evaluated based on selected transparency rules. The transparency rules can indicate which fragments should be kept and/or discarded to provide desired transparency visualizations. In the example of FIG. 24, an example set of transparency rules may indicate that the second fragment 2514 and the third fragment 2516 should be discarded to avoid cluttering a visualization with representation of the internal surfaces, for example by providing a rule based on relationships between multiple points (e.g., a rule to discard any point having a point both closer and further from the camera 2504 to said point). Step 2404 can also include associating an opacity level (e.g., completely opaque, 60% opacity, 20% opacity, complete transparency, etc.) to different fragments. Various rules are implementable in step 2402 depending on the particular visual effects desired to be achieved using process 2400. Advantageously, such rules can be applied using results of step 2402 without prior knowledge or data stored with the object 2500 indicating which surfaces are front, back, interior, etc. In some embodiments, after passing through the evaluation of step 2404, fragments area appended to a linked listed for an order independent transparency algorithm.

At step 2406, fragments are ordered. Ordering the fragments can include determining the order in which the fragments are intersected by a ray. In the example of FIG. 25, step 2406 can include determining that the ray 2502 first intersects the first fragment 2510, then intersects the second fragment 2512, then intersects the third fragment 2514, then intersects the fourth fragment 2516, for example based on a determined depth of the fragments along the ray 2502. Step 2406 can determine that the first fragment 2510 is closer to the virtual camera 2504 than the last segment 2516. Various sorting and/or ordering operations can be executed in step 2406.

At step 2408, a pixel color is determined based on blending fragments based on ordering. For example, based on the transparency rules evaluated in 2404, a set of relevant fragments can be provided, with each fragment provided with an associated color (with the color being further based on a transparency level, in various embodiments). Based on the order of such fragments (e.g., whether a first fragment is in front of or behind a second fragment as determined in step 2406), the associated colors can be blended (combined, merged, etc.) to provide a color for the pixel associated with a ray. The color can be based on a distance between entry exit points of an object, for example. By repeating such operations for different rays cast across an object according to teachings described elsewhere herein, a visualization with transparent features can be provided, thereby enabling a user to see objects internal to other objects, internal features of objects, etc. in various embodiments.

Various visualizations can thereby be provided. With reference to FIG. 25, if fragment 2510 is assigned full opacity, the object 2500 will appear as a solid object without visualization of the other fragments. As another option, all fragments 2510-2516 may be assigned less than full opacity (e.g., 60% opacity), in which case the visualization can provide the ability to see all surfaces. As another option, the first fragment 2510 and the fourth fragment 2516 can be provided with partial transparency, while the second fragment 2512 and/or the third fragment 2514 is/are shown as opaque, facilitating a view of the internal structure of the object 2500. As another option, the fourth fragment 2516 can be provided as opaque while the first fragment 2510 is provided as transparent (e.g., 60% opacity), thereby facilitating a view of the back surface of the object 2500. Various such examples are possible, for example selectable by a user to facilitate viewing of features of interest in the visualizations contemplated by the present disclosure.

Such processes can also be used to generate shadows to be included in visualization of a surgical scene. For example, in some embodiments, a depth map is created using accumulated buffers to generate depth maps from the perspective of a light source to various surfaces within the scene in a first pass. Rendering can then be performed from a viewpoint using the techniques above, where shading is then accomplished by comparing the sorted depth values of each sell in the accumulated buffer from depth values created in the first pass, which provides a determination as to whether a fragment in the scene is in shadow relative to the light source. In some embodiments, a perspective shadow mapping technique which transforms the scene into the camera perspective before generating shadow maps. In some embodiments, a cascaded shadow mapping approach is performed which divides the view frustum into multiple segments or cascades, each with own shadow maps, which can advantageously provided for high-resolution shadow near to the camera and progressively low-resolution shadows for further-away objects.

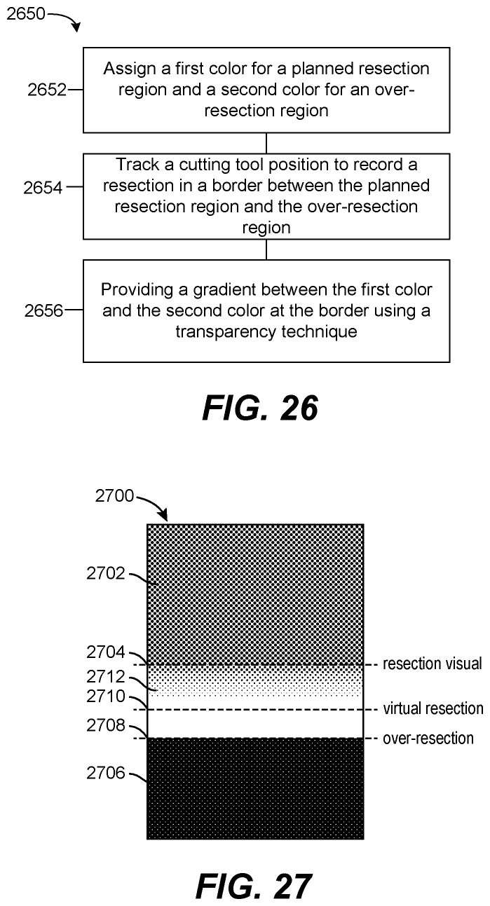

Referring now to FIG. 26, a flowchart of a process 2650 is shown, according to some embodiments. The process 2650 can be executed by one or more processors of instructions stored in non-transitory computer-readable media, e.g., by the system 10 for example by or using the navigation system 22 (e.g., by the GPU 28). The process 2650 can be executed in combination with any of the various other processes and approaches described herein.

At step 2652, a planned resection region is assigned a first color and an over-resection region is assigned a second color. Step 2652 can thus facilitate differentiating a planned resection from a region which has been over-resected by a surgical tool. For example, the first color may be blue and the second color may be yellow or red. A third color may be assigned for a bone model (e.g., in regions outside the planned resection or over-resection) and/or other objects to be represented in a visualization. Step 2652 can include establishing the planned resection, for example by planning an implant position and using the implant position to generate a volume to be resected in order to enable placement of an implant in the planned implant placement. The over-resection region can be defined as portions of the bone model which intersect an object representing a completed resection without intersecting the planned resection region, in accordance with the teachings above.

At step 2654, a tool position is tracked to record a resection in a border between the planned resection region and the over-resection region. For example, by tracking a tool position and using a shape-sweeping technique as described above, a representation of a resection as completed by a cutting tool can be determined. In the context of step 2654, such a representation of the resection lies at least partially in a border between the planned resection region and the over-resection region, for example within a threshold distance of the planned resection and the original bone model, as will be described below with reference to FIG. 27.

At step 2656, a gradient is provided in a visualization between the first color and the second color at the border using a transparency (or translucency) technique. Step 2656 can include generating live updates to a visualization to guide completion of a resection, as described above, together with representing a surface falling in the border between the planned resection region and the over-resection region with a color gradient, thereby providing guidance to a user as to the degree to which a surface of a tracked resection is approaching or exceeding the intended resection.

In some embodiments, the transparency (or translucency) technique executed at step 2656 is according to the teachings of FIGS. 24-25. In some embodiments, the color of a CSG fragment at a position p₁ along the viewing ray can be calculated as: C=F (p₁, p₂, φ), where Fis a function that returns a color value C=(R, G, B, a) based on the position p₁ of the fragment, position p₂ of the next visible fragment along the viewing ray and material parameters o (e.g., relating to opacity, translucency, etc. of a given material). To achieve a translucency or gradient effect, the following function can be used in step 2656: C(p₁)=C₁(1−e^{-μd}), where d is the distance between points p₁ and p₂ (d=length(p₁-P₂)) and C₁ and μ are material parameters. Such a technique can be implemented by using a point on a surface of an over-resection region intersected by a viewing ray as position p₂ for comparison to the position p₁ along the viewing ray that intersects a surface of the tracked resection, such the color is determined as a function of the distance between such points along the ray. Such an approach can enable a gradient color representation that provides a user with enhanced visualization of margins along the border of the planned resection, thereby enhancing understanding and guidance of resection at the margin.

Referring now to FIG. 27, a diagram 2700 of gradation between a planned resection region and an over-resection region is shown, according to some embodiments. As show in FIG. 27, the first color is provided in the planned resection region 2702 up to a boundary 2704 of the resection visualization and the second color is provided in an over-resection region 2706 beyond an over-resection boundary 2708, with the planned resection region 2702 and the over-resection region 2706 spaced apart across a surface 2710 (e.g., a surface 2710 representing the planned resection intended to be completed at maximal precision) and thus providing a border region 2710 between the planned resection region 2702 and the over-resection region 2706. A color gradient according to process 2650 is illustrated in the border 2710, with the color gradient being from the first color to the second color and extending across the border region 2710 between the boundary 2704 of the planned resection region 2702 and the over-resection boundary 2708. The color gradient may be a substantially-continuous change in color from the first color to the second color, such that an outer extent of a completed resection is provided with a range of coloration to illustrate how well different areas of the resection match the originally-planned resection (e.g., showing deviation from surface 2710). Such color gradation can advantageously provide significantly more information as compared to, for example, making a sudden switch between the first color and the second color at the surface 2710 or providing a solid, third color in the border region 2712. The teachings of FIG. 26-27 can be implemented as part of any of the processes, systems, etc. described elsewhere herein.

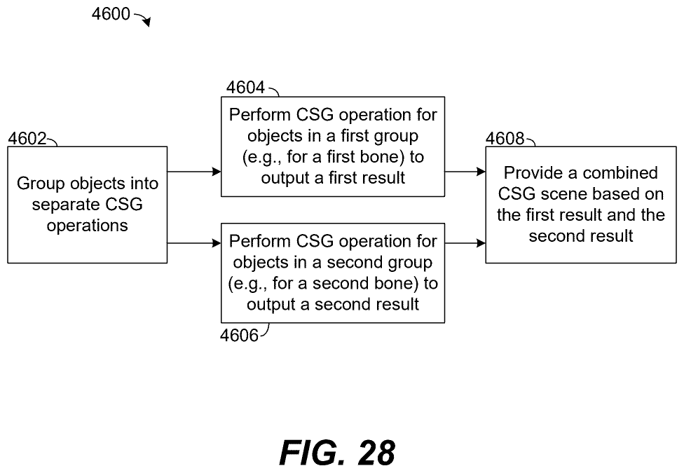

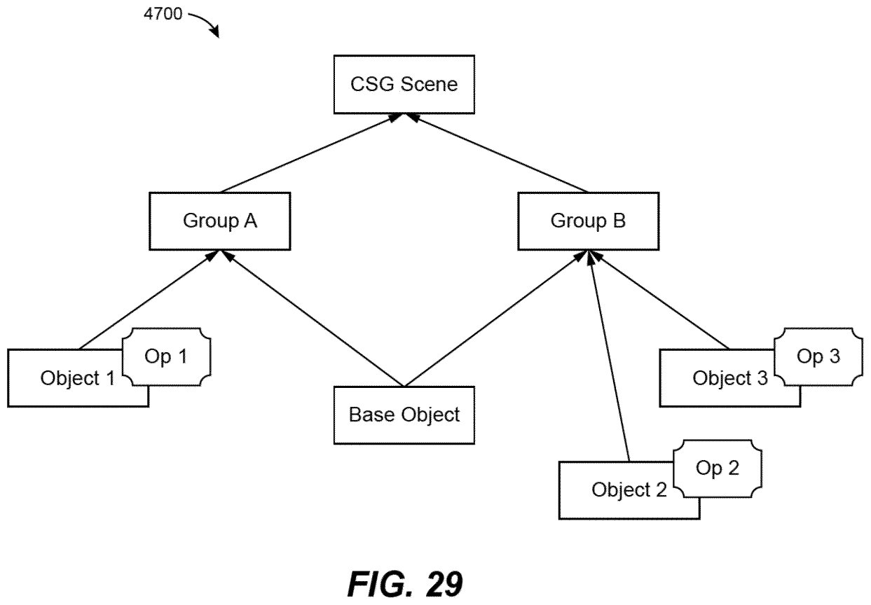

Referring now to FIGS. 28-29 approaches for grouping CSG operations are shown, according to some embodiments. FIG. 28 shows a process 4600 for providing a combined CSG scene, while FIG. 29 shows a diagram 4700 illustrating an example grouping of CSG operations. Process 4600 can be executed by one or more processors executing instructions stored on one or more non-transitory computer-readable media, where the one or more non-transitory computer-readable media store instructions, programming, etc. that, when executed by the one or more processors, cause the one or more processors to perform the operations of process 4600. Process 4600 can be executed together with, using, as part of, etc. any of the other processes described herein. Process 4600 can be executed by the system 10, for example by the CPU 26 and/or GPU 28.

In some scenarios, grouping of CSG operations can be advantageous. In the context of surgical modeling of a joint for example, certain CSG objects may be associated with a first bone of the joint (e.g., a model of a tibial resection is associated with the tibia) while other CSG objects may be associated with a second bone of the joint (e.g., a model of a femoral resection is associate with the femur). However, a challenge exists in avoiding unwanted interactions between objects associated with a first bone and a model of a second bone, for example, for example an interaction which may result in subtraction of a portion of the second bone model based on an object representing a resection to be executed on a first bone. The computer visualization techniques of FIGS. 28-29 can address such technical challenges.

At step 4602, objects are grouped into separate CSG operations. For example, objects associated with a first bone (e.g., a first bone model, a first implant model of an implant to be implanted on the first bone, a first resection model of a resection to be executed on the first bone) are grouped in a first group while objects associated with a second bone (e.g., a first bone model, a second implant model of an implant to be implanted on the first bone, a second resection model of a resection to be executed on the second bone). Various other groups can be provided depending on the scenario presented, the physical relationship between different object being modeled, etc., in various embodiments. As illustrated in FIG. CC, an Object 1 and a Base Object are provided into Group A, while the Base Object, an Object 2, and an Object 3 are provided into Group B. The Base Object can be a structure, background object, coordinate frame, etc. common to both groups.

At step 4604, a CSG operation is performed for objects in the first group (e.g., objects associated with a first bone) to output a first result. The CSG operation for the first group can be performed independent of the objects in the second group. At step 4606, a CSG operation is performed for objects in the second group (e.g., objects associated with a second bone) to output a second result. The CSG operation for the second group can be performed independent of the objects in the first group. In the example of FIG. 7, a first CSG operation is performed for Group A using the objects in Group A, while a second CSG operation is performed for Group B using the objects of Group B.

At step 4608 a combined CSG scene is provided based on the first result and the second result. The first result can be a model corresponding to the first group (e.g., corresponding to a first bone) while the second result can be a model corresponding to the second group (e.g., corresponding to a second bone), while the combine CSG scene shows both models in a combined visualization. The combined visualization can be provided as an addition of the different group results, such that both are shown (e.g., with some portions obstructed from view depending on relative positions and the viewpoint of a virtual camera). In the context of FIG. CC, for example, results of CSG operations in both Group A and Group B are provided, without performing a CSG operation directly involving both Object 1 and either Object 2 or Object 3. Unintended or incorrect results which may otherwise occur from a CSG operation involving both Object 1 and either Object 2 or Object 3 are thereby avoided. The approach of FIGS. 28-29 can also reduce computational complexity (e.g., and thus increase computer image generation speed) in calculating the final CSG scene as compared to an approach in which a CSG operations is provided for all objects without grouping and involving a larger number of comparisons, rules, algorithms, etc. as may then be needed without the teachings of FIGS. 28-29.

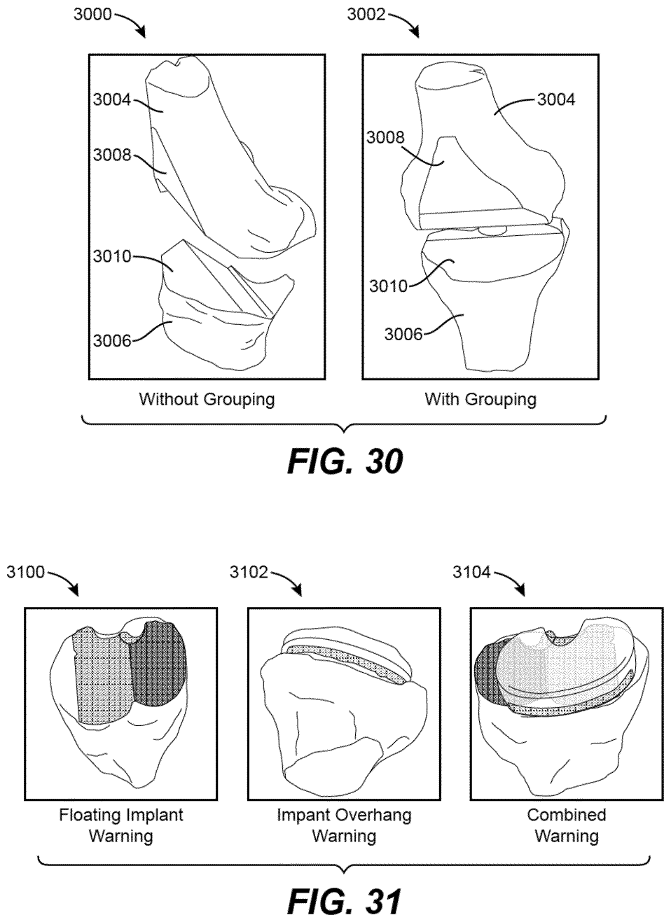

Referring now to FIG. 30, a first example result of the grouping approach of FIGS. 28-29 is shown, according to some embodiments. In particular, FIG. 30 includes a first illustration 3000 showing a visualization of a joint in which bone resections and implant models relative to bone models without grouping, while a second illustration 3002 showing a similar visualization except generated using the grouping approach of FIGS. 28-29. In particular, in the example shown, a femur 3004 and tibia 3006 are shown together with placement of a femoral implant component 3008 and a tibial implant component 3010. In order for the femoral implant component 3008 and the tibial implant component 3010 to be visible (e.g., not hidden inside the representation of the femur 3004 and the tibia 3006), subtraction of planned femoral resection and of a planned tibial resection from the visualizations 3000, 3002 are performed.

However, without grouping as in the first visualization 3000, subtraction of the planned femoral resection from the first visualization 3000 causes part of the tibia 3006 and tibial implant component 3010 to be subtracted from the first visualization 3000 and subtraction of the planned tibial resection causes part of the femur 3004 and the femoral implant component 3008 to be subtracted from the first visualization 300. This results in the blank region 3012 of the first visualization 3000 between the femur 3004 and the tibia 3006.

Advantageously, the second visualization 3002 is performed using a grouping technique as in FIGS. 28-29 whereby the tibial objects (tibia 3006, tibial implant component 3010, and tibial resection to be subtracted) are grouped separately from a group of the femoral objects (femur 3004, femoral implant component 3004, and the planned femoral resection to be subtracted). Such grouping prevents subtraction of the femoral resection from affecting the tibia-side representation and prevents subtraction of the tibial resection from affecting the femoral-side representation, thereby resulting in the second visualization 3002 which provides useful visibility of articular surfaces of the joint.

Referring now to FIG. 31, another example enabled by the grouping techniques herein are shown, according to some embodiments. In particular, by grouping objects for a first type of warning (shown as a floating implant warning in box 3100) separately from objects for a second type of warning (shown as an implant overhang warning in box 3102), separate groups and CSG operations can first be executed to handle the first type of warning and the second type of warning. Then, the resulting objects can be combined to provide a combined warning visualization as shown in box 3104 as shown in FIG. 31. The techniques herein can thereby provide for display of rich information including multiple warnings (e.g., displayed during implant planning) or other enhancements which are associated with logic which might otherwise obscure aspects of a visualization or prevent such visualization from being properly generated.

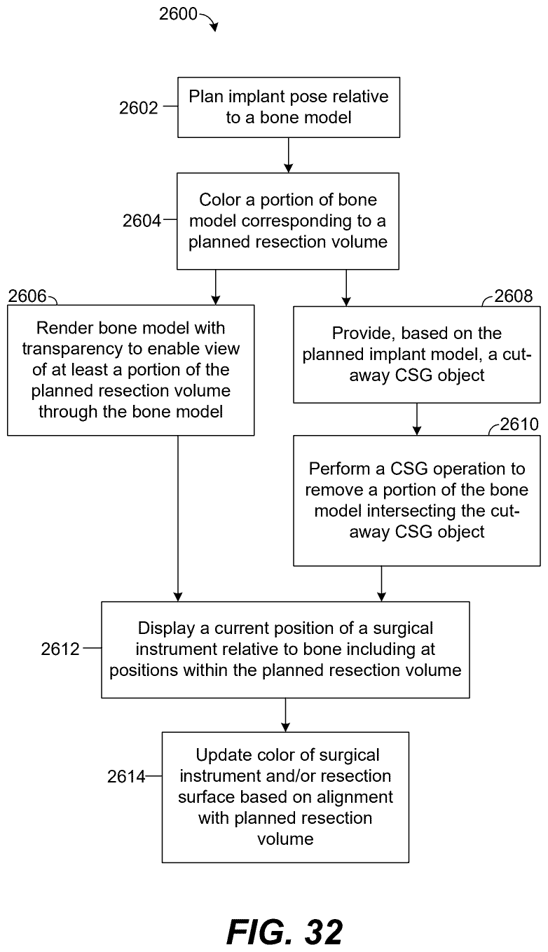

Referring now to FIG. 32, a flowchart of a process 2600 is shown, according to some embodiments. The process 2600 can be executed by one or more processors executing instructions stored on one or more non-transitory computer-readable media, where the one or more non-transitory computer-readable media store instructions, programming, etc. that, when executed by the one or more processors, cause the one or more processors to perform the operations of process 2600. Process 2600 can be executed together with, using, as part of, etc. any of the other processes described herein. Process 2600 can be executed by the system 10, for example by the CPU 26 and/or GPU 28.

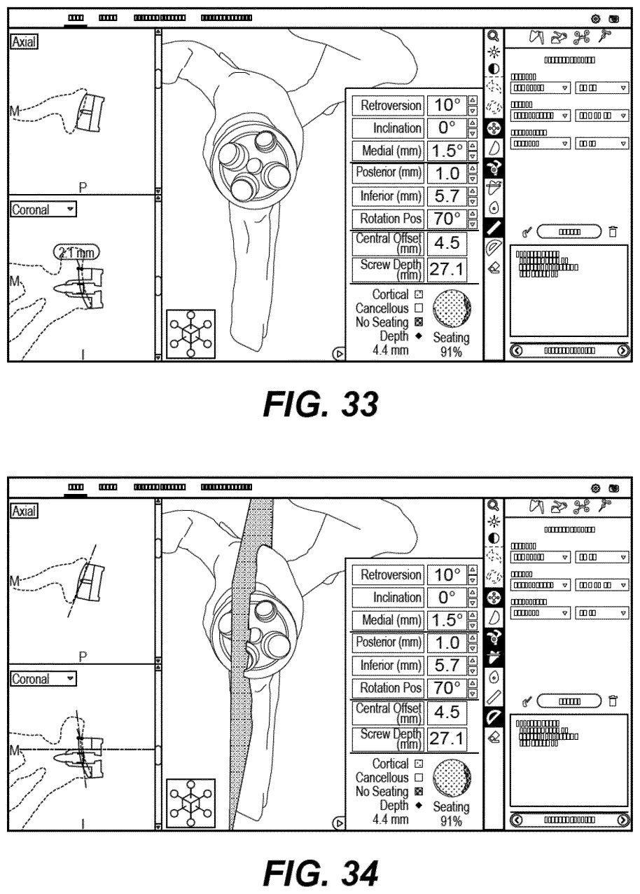

At step 2602, an implant position is planned relative to a bone model. The implant position can be planned via a graphical user interface, for example as shown in FIGS. 33-35. FIG. 33 shows an interface where a pose (i.e., position and orientation) (and, in some embodiments, a size, type, etc.) of an implant component is planned relative to a bone model. Step 2602 can include displaying the implant model relative to the bone model, while allowing a user to adjust the implant position via interaction with the graphical user interface. In some embodiments, planning the implant position can be performed automatically by the system 10, for example by optimizing the implant position according to clinical criteria and/or surgeon-selected targets or ranges for implant pose and/or resulting joint geometry.

As illustrated in FIGS. 33-36, various views and display objects can be provided (or selectively hidden) via a graphical user interface. FIG. 33 shows an implant model in position relative to a bone model, for example with the bone model virtually resected as needed to receive the implant model in the planned position. FIG. 33 also includes a graphic showing a surface map showing which portions of the implant model are seated on (contacting, mated against, abutting, etc.) the bone model.

As shown in FIG. 34, a user has selected an icon which causes the graphical user interface to additionally show an anatomic plane intersecting the bone model and implant model, for example to facilitate a user in adjusting implant position relative to the anatomic plane. The anatomic plane is shown as being translucent, such that portions of the bone model and implant model can be seen through the anatomic plane, for example according to the teachings of FIGS. 24-25.

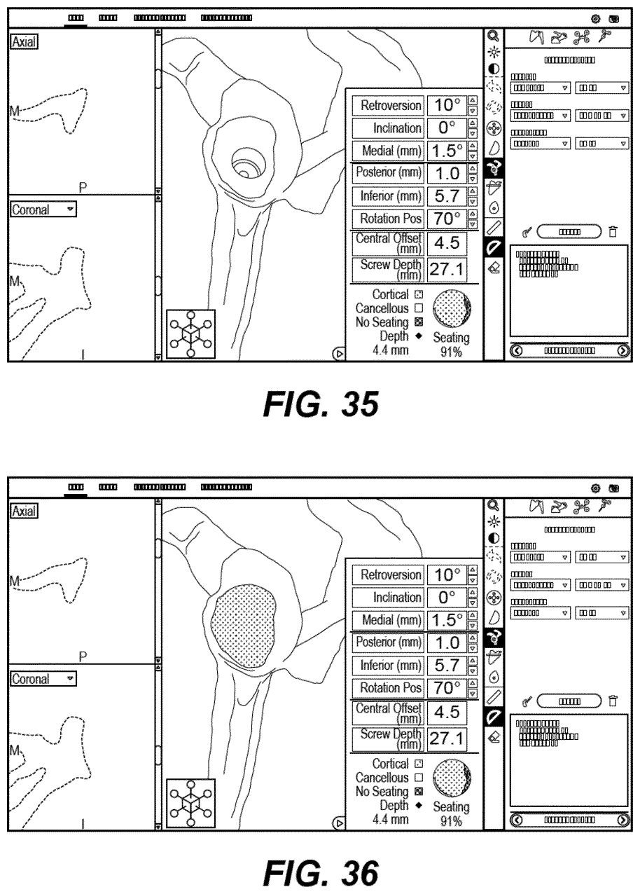

As shown in FIG. 35, a user has selected icons to hide (omit from displaying) the anatomic plane and the implant model, such that the bone model is shown, in particular such that the bone model is shown as if a planned resection has been completed to prepare the bone to receive the implant in the planned pose. As shown in FIG. 35, the planned resection includes a resection volume that extends into the bone model along a post, projection, etc. of the planned implant. FIG. 30 also shows an implant planning view and described in further detail below.

In each of FIGS. 33-36, the graphical user interface includes selectable buttons (arrows, etc.) for adjusting implant position, selection of which causes movement of the implant model relative to the bone model and an update of any of the visualization shown. FIGS. 33-36 also show cross-sectional CT or x-ray style visualizations to assist in implant planning. Once the user is satisfied with the displayed planned pose of the implant model relative to the bone model, an option on the graphical user interface can be selected to advance to a next stage of the surgical workflow.

Still referring to process 2600 of FIG. 32, at step 2604 a portion of the bone model corresponding to a planned resection volume is colored differently than a remainder of the bone. For example, as shown in FIG. 30, an opaque view including a region of bone planned for resection colored to be distinguishable from the bone model is shown. For example, the material to be resected from the bone model can be colored in blue, as shown in FIG. 36, while a remainder of the bone model is a different color (e.g., grey, beige, etc.). FIG. 36 can thereby provide a visualization of a surface of the bone model that will be modified when a bone resection is executed. Step 2604 can be executed by performing a CSG operation to find an intersection of the implant model and the bone model and coloring the intersection as a different color than a remainder of the bone model.

Step 2604 can include advancing a surgical workflow from an implant planning stage to a bone preparation stage in which resection of the planned portion of the bone is facilitated. In some embodiments, bone preparation is provided in multiple stages to complete resection of the entire region planned for resection to prepare a bone to receive an implant in the planned position from step 2602.

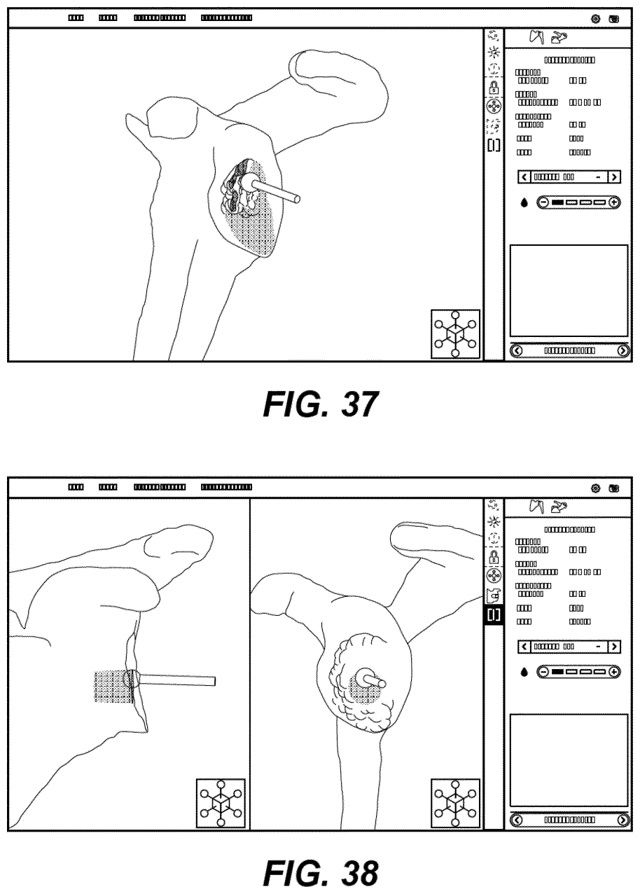

As shown in FIG. 37, for example, a visualization of a surface region of the bone to be sculpted can be provided while a surgical instrument is also shown in the graphical user interface in its real-time position relative to the bone. Haptic feedback can be provided by the system 10 to constrain the surgical instrument to the surface region. As shown in FIG. 37, and using teachings described elsewhere herein, the visualization can be updated in real-time (or near real-time) so as to remove portions from the bone model when the surgical instrument resections such portions of the real bone in real space. A warning color (e.g., yellow) can be provided in regions where the bone was resected at or past a boundary of the planned resection, while a base color of the bone model can be provided to indicate successful resection in accordance with a plan and another color (e.g., blue) can be provided to show a volume of bone still to be resected to complete a first stage of the bone resection procedure. In particular, FIG. 37 shows an example of a glenoid surface preparation stage of a bone preparation stage of a surgical workflow using the system 10. Upon completion of such a first bone preparation stage, process 2600 can advance to step 2606 and/or step 2608. In other embodiments, no such first bone preparation stage is provided and step 2606 and/or step 2608 are executed immediately after completion of implant planning.

Still referring to FIG. 32, process 2600 can facilitate execution of surgical resections which protrude into a bone model using a transparency technique (via step 2606) and/or using a cut-away visualization technique (via steps 2608). Either or both techniques can be executed, for example in a combined visualization approach and/or such that a user can select between (toggle between) different types of views (e.g., by selecting icon on a graphical user interface).

At step 2606, the bone model is rendered with transparency to enable view of at least a portion of the planned resection volume through the bone model. A surgical instrument (e.g., cutting tool such as a burr) can also be visualized as part of the combined visualization, for example so as to be visible at least in part through a portion of the bone model. Step 2606 can be executed using the teachings of FIGS. 24-25, for example.

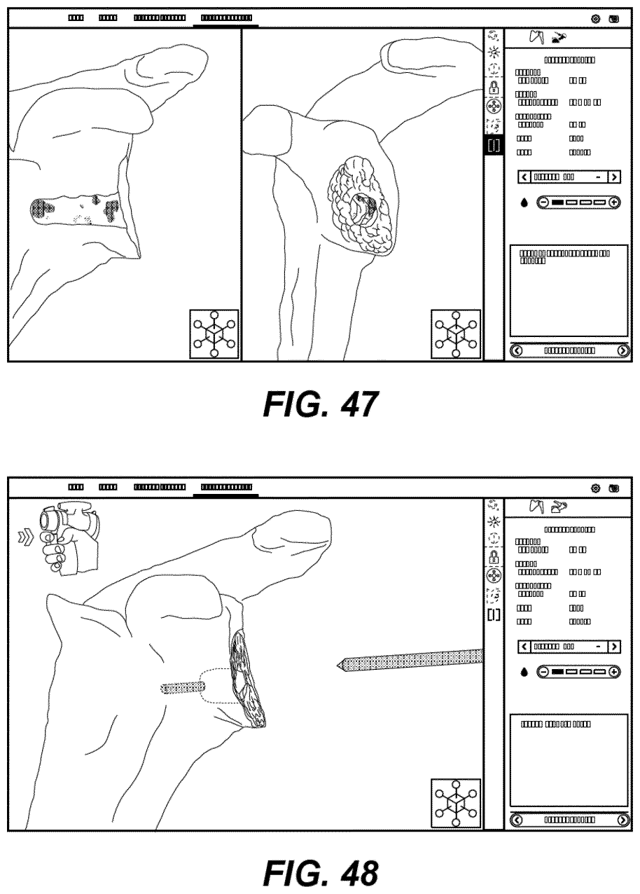

FIG. 38 shows an example of a graphical user interface that can be provided according to step 2606. A shown, in the view provided on a left side of the graphical user interface in the example shown, a colored planned resection volume is visible through a portion of the bone model which is between the planned resection volume and a view point of a virtual camera viewing the scene. A position of a surgical instrument is also shown relative to the bone model and planned resection volume. Such a visualization provides depth information to a viewer, so as to facilitate movement of the surgical instrument in real space to resect real bone in accordance with the planned resection volume. The graphical user interface of FIG. 38 includes options to change the perspective of view of the virtual models shown (e.g., jump to a different axis and/or freely rotate the models), in response to which the transparency operations can be updated (e.g., using the teachings disclosed above) so as to provide translucency through a portion of the bone model closest to a virtual camera viewing the scene. FIG. 47 also shows an example translucent view, for example following completion of a bone resection. Step 2606 thereby enables visualization and navigation of a bone resection from any different perspective view of a bone model, including views from which the planned resection volume would not be visible without the transparency features provided according to step 2606.

As an additional or alternative option to providing navigation of resections at depths into a bone, a cut-away view can be provided via steps 2608 and 2610 of process 2600. At step 2608, a cut-away CSG object is provided based on the planned implant pose (e.g., based on the planned pose for an implant from step 2602). The cut-away CSG object can be a three-dimensional volume with a first end aligned with an shaped to match a shape of a post, projection, etc. of a planned implant and extending away from the implant model in a first direction in a widening shape. For example, the cut-away CSG object can have a cake-slice shape, with a tip shaped to match and coincident with a curvature of a portion of the implant model, having sides extending along planes tangential to the portion of the implant model (e.g., such sides being at an angle between forty-five degrees and ninety-degrees, for example sixty degrees), and, in some embodiments, having a bottom surface aligned with a depth of the of the implant model into the bone model.

In some embodiments, the system 10 stores and/or accesses a library (datastore) of implant models for different implant sizes, types, etc. which can also include a cut-away CSG object associated with each implant model. A spatial relationship between a cut-away CSG object and its associated implant model can be stored with the models in said library. In some embodiments, a rotation of the cut-away CSG object relative to (e.g., about an axis of) an associated implant model is predefined (i.e., static), selected based on an anatomical plane or axis of a patent (e.g., so as to always point medially, laterally, posteriorly, anteriorly, etc. from the implant model), and/or dynamically updated based on a view point of a virtual camera viewing a scene generated according to the teachings herein.

At step 2610, a CSG operation is performed to remove a portion of the bone model intersecting the cut-away CSG object. In particular a subtraction can be performed which provides a resulting model which includes the bone model without the portion thereof intersected by the cut-away CSG object but while still showing a planned resection volume. The cut-away CSG object itself is not shown (e.g., is invisible).

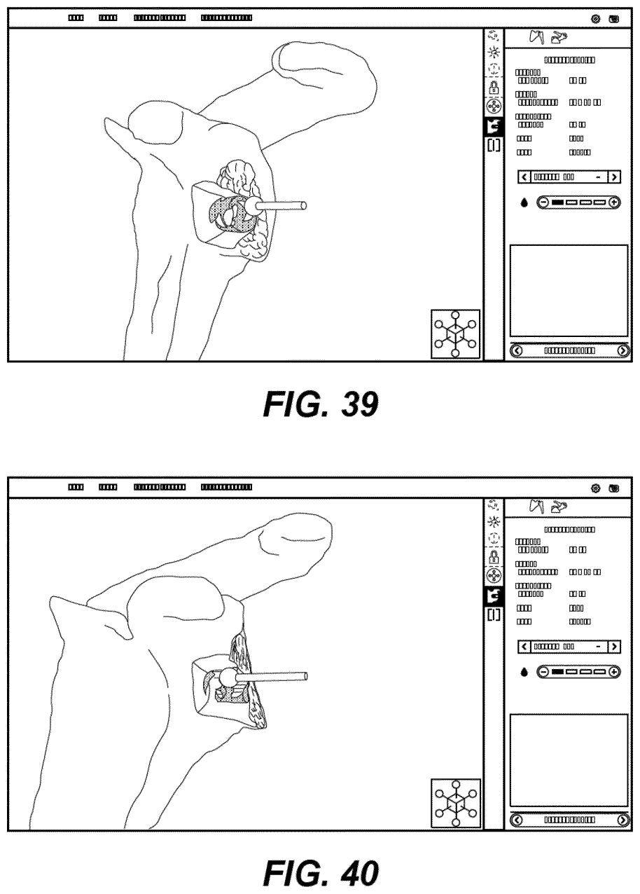

Example results of step 2610 are shown in FIGS. 39-46, according to various example embodiments. The graphical user interfaces of FIGS. 39-46 show a bone model with space intersecting a cut-away CSG object removed, with a planned resection volume provided in such space so as to now be visible via the region corresponding to the cut-away CSG object. That is, visibility is provided through the cut-away CSG object to the planned resection volume.

Still referring to process 2600 of FIG. 32, the examples of FIGS. 38-47 also illustrate execution of step 2612 and step 2614. At step 2612, a current pose of a surgical instrument is displayed together with the visualization generated in preceding steps of process 2600, such that the pose of the surgical instrument relative to the planned resection volume can be seen on the graphical user interface. At step 2614, coloration of the resection volume and/or the surgical tool can be updated as the bone resection is executed to provide information on progress and accuracy of completion of the bone resection.

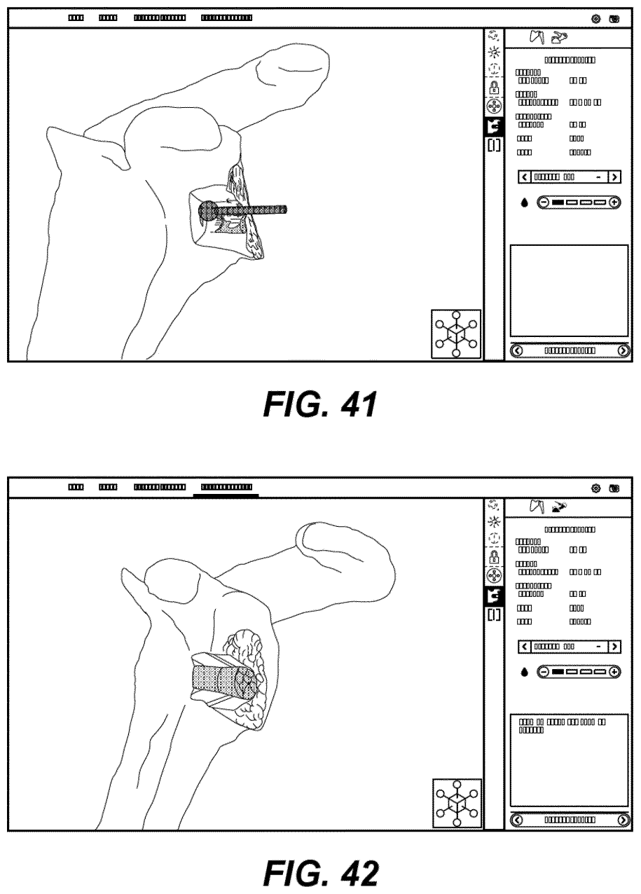

For example, as illustrated in FIGS. 39-41, as the surgical tool is passed within the planned resection volume, the surgical tool can be provided in a first color (e.g., green) and the planned resection volume can be updated from a solid or translucent object (e.g., blue) to remove (e.g., hide, delete, make invisible) portions thereof which have been resected by the tool (e.g., which have been passed through by a tracked pose of the surgical instrument). Accordingly, remaining portions of the planned resection volume still to be resected can be shown, for example as in FIGS. 39-41, while the cut-away visualization provided by steps 2608-2610 facilitates a user in seeing the tool at a depth relative to such remaining portions (including at least partially behind such remaining portions), which can facilitate completion of a bone resection. Additionally, in some embodiments, as illustrated in FIG. 41, the surgical instrument and/or a resected bone surface can be colored in a warning color (e.g., yellow) in response to the surgical instrument reaching into or beyond a boundary of the planned resection volume, indicating to a user that the surgical instrument should not be pushed further in a corresponding direction.

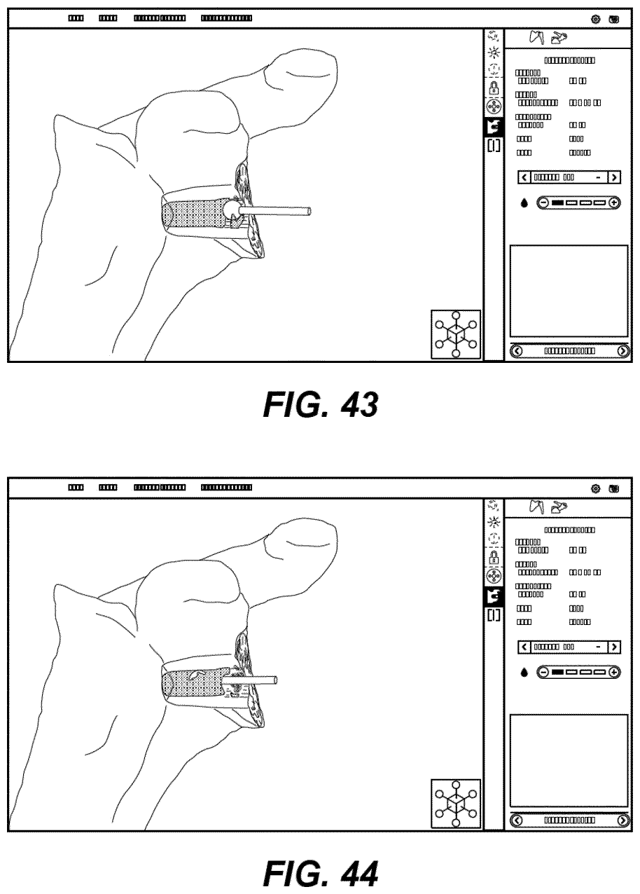

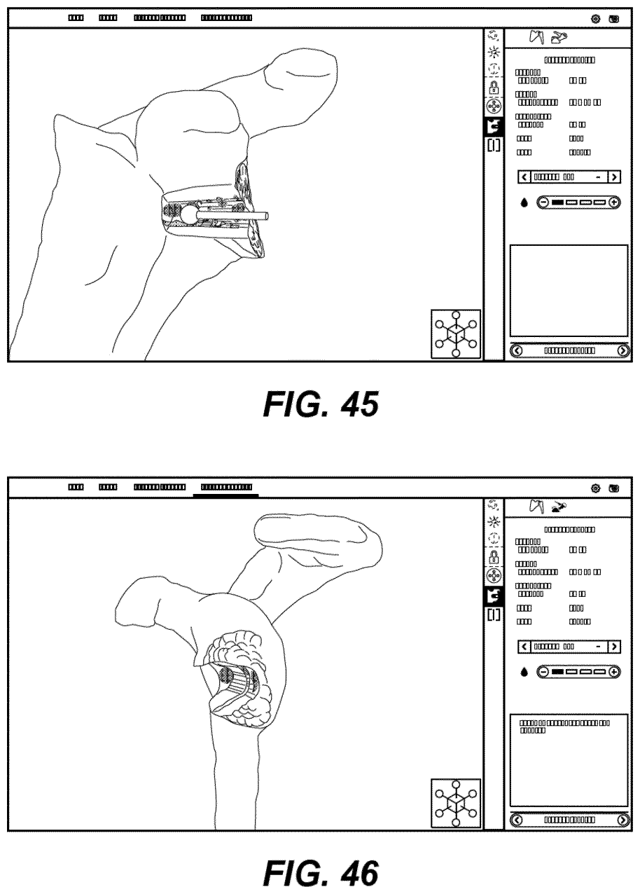

FIGS. 42-45 shown another example embodiment, including a series of a visualizations that can be shown on a graphical user interface as a bone sculpting or resection stage moves from a beginning states (where the full, planned resection volume is visible as in FIG. 42) through partial resection stages where the cutting tool is visualized as extending into the resection volume (e.g., as in FIGS. 43-44) and as the bone resection is substantially complete such that the resection volume has been substantially removed from the visualization with some regions highlighted to shown actual resection depth relative to the planned resection volume (e.g., as in FIG. 45). FIGS. 46-47 show further views available to a user following completion of a bone resection operation, including a cut-away view (FIG. 46) and a transparency view (FIG. 47) generated in accordance with the teachings above and showing results of a bone resection.

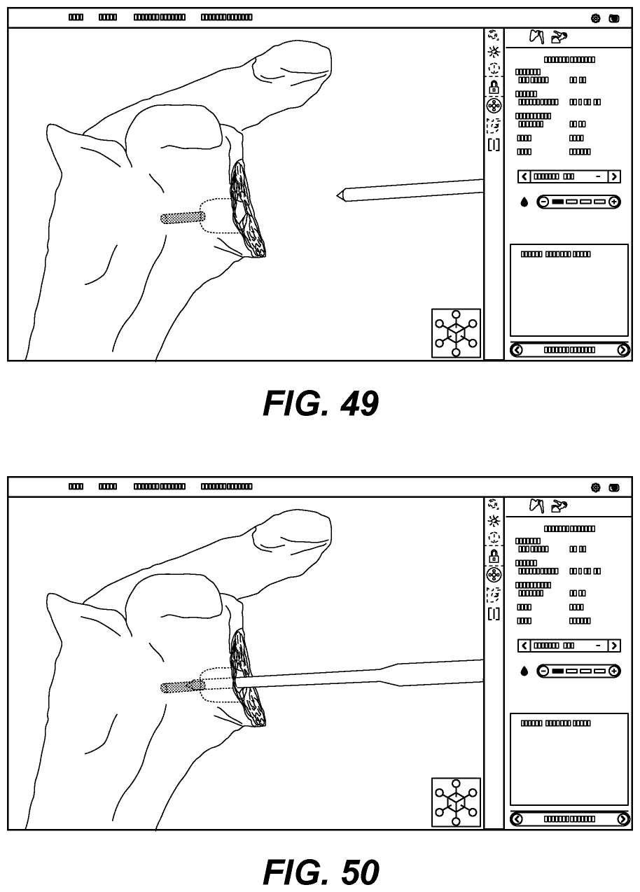

As yet further examples, FIGS. 42-44 show additional transparency views in which a planned screw hole (e.g., to be pre-drilled as a pilot hole) is shown inside a bone model using a transparency technique (e.g., according to step 2606). In the example of FIG. 48, a virtual surgical tool (e.g., drill) is shown in a first color (e.g., blue, grey) when not aligned with the planned screw hole. In FIG. 49, the virtual surgical tool is changed in color (e.g., to green) to indicate that a corresponding surgical tool in real space has been aligned with the planned screw hole. FIG. 50 shows a transparent view in which the virtual surgical tool can be seen extending into the bone and into the planned screw hole to complete drilling of the hole. Without the transparency techniques taught herein, such a visualization would not be possible and the graphical user interface could shown only an opaque bone surface obstructing any view of the surgical tool's progress in extending into the planned screw hole.

Process 2600 and the various example interfaces provided herein thereby provide substantially-improved visualizations for surgical navigation, for example for use in robotically-assisted surgical procedures such as those including controlling a robotic device to provide haptic feedback based on the planned resection volumes described above.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

What is claimed is:

1. A method, comprising:
providing a bone model and a surgical plan defining a planned implant pose to the bone model;
providing, based on the planned implant pose, a planned resection volume and a cut-away object;
generating a visualization for surgical navigation by:
subtracting, from the bone model, a region of intersection between the bone model and the cut-away object; and
including a visualization of the planned resection volume; and
dynamically updating the visualization of the planned resection volume to show progress in completing a resection by tracking movement of a surgical instrument completing the resection.

2. The method of claim 1, wherein providing the planned resection volume and the cut-away object comprise retrieving the planned resection volume and the cut-away object from a library based on a selected size of an implant to be implanted in the planned implant pose.

3. The method of claim 1, comprising orienting the cut-away object relative to the planned resection volume based on an anatomic axis or plane of the bone model.

4. The method of claim 1, wherein dynamically updating the visualization of the planned resection volume comprises:
providing a remainder of the visualization of the planned resection volume to be resected in a first color;
coloring an over-resected region beyond the planned resection volume in a second color; and
providing a resected surface between the over-resected region and the visualization of the planned resection volume with a color gradient from the first color to the second color.

5. The method of claim 1, further comprising:
including, with the visualization, a virtual model of the surgical instrument by:
coloring the surgical instrument in a first color prior to alignment with the planned resection volume;
coloring the surgical instrument in a second color while aligned with the planned resection volume; and
coloring the surgical instrument in a third color when the surgical instrument reaches a boundary of the planned resection volume or deviates from the planned resection volume.

6. A computer visualization method for surgical planning and/or navigation, comprising:
grouping constructive solid geometry (CSG) objects associated with at surgical scene into a first group and a second group;
performing a first CSG operation using CSG objects in the first group independent of CSG objects in the second group;
performing a second CSG operation using CSG objects in the second group independent of CSG objects in the first group; and
visualizing a combined CSG scene using a first result of the first CSG operation and a second result of the second CSG operation.

7. The computer visualization method of claim 6, wherein the first group corresponds to a first bone of a joint and the second group corresponds to a second bone of a joint.

8. The computer visualization method of claim 6, wherein the first group and the first CSG operation are associated with a first type of implant planning warning and wherein the second group and the second CSG operation are associated with a second type of implant planning warning.

9. The computer visualization method of claim 6, the CSG objects comprise a bone model, a planned resection model, and a cut-away object.

10. The computer visualization method of claim 9, wherein the first CSG operation comprises subtracting, from the bone model, a region of intersection between the bone model and the cut-away object.

11. The computer visualization method of claim 10, wherein visualizing the combined CSG scene comprises dynamically updating a visualization of the planned resection volume to show progress in completing a resection by tracking movement of a surgical instrument completing the resection.

12. The computer visualization method of claim 11, wherein dynamically updating the visualization of the planned resection volume comprises:
providing a remainder of the visualization of the planned resection volume to be resected in a first color;
coloring an over-resected region beyond the planned resection volume in a second color; and
providing a resected surface between the over-resected region and the visualization of the planned resection volume with a color gradient from the first color to the second color.

13. A surgical robotics system, comprising:
a robotic device configured to guide a surgical instrument in accordance with a planned resection;
a display screen; and
a control system communicable with the robotic device and the display screen and programmed to perform operations comprising:
providing a bone model and a surgical plan defining a planned implant pose to the bone model;
providing, based on the planned implant pose, a planned resection volume and a cut-away object;
causing the display screen to present a visualization for surgical navigation by:
subtracting, from the bone model, a region of intersection between the bone model and the cut-away object; and
including a visualization of the planned resection volume; and
dynamically updating the visualization of the planned resection volume to show progress in completing a resection by tracking operation of the robotic device while the robotic device guides the surgical instrument in completing the resection.

14. The surgical robotics system of claim 13, wherein the surgical instrument is a burr and the bone model is a model of a portion of a shoulder joint.

15. The surgical robotics system of claim 13, wherein subtracting, from the bone model, the region of intersection between the bone model and the cut-away object enables a representation of the burr to be visible in the visualization through the bone model via a space corresponding to the cut-away object.

16. The surgical robotics system of claim 13, wherein providing the planned resection volume and the cut-away object comprise retrieving the planned resection volume and the cut-away object from a library based on a selected size of an implant to be implanted in the planned implant pose.

17. The surgical robotics system of claim 13, wherein the operations comprise orienting the cut-away object relative to the planned resection volume based on an anatomic axis or plane of the bone model.

18. The surgical robotics system of claim 13, wherein the computing system is programmed to dynamically update the visualization of the planned resection volume by:
providing a remainder of the visualization of the planned resection volume to be resected in a first color;
coloring an over-resected region beyond the planned resection volume in a second color; and
providing a resected surface between the over-resected region and the visualization of the planned resection volume with a color gradient from the first color to the second color.

19. The surgical robotics system of claim 13, wherein the control system is further programmed to cause the display screen to present a planning interface for planning the planned implant pose relative to the bone model, wherein the control system is programmed to generate the planning interface by:
grouping constructive solid geometry (CSG) objects associated with at surgical scene into a first group and a second group;
performing a first CSG operation using CSG objects in the first group independent of CSG objects in the second group;
performing a second CSG operation using CSG objects in the second group independent of CSG objects in the first group; and
visualizing a combined CSG scene using a first result of the first CSG operation and a second result of the second CSG operation.

20. The surgical robotics system of claim 19, wherein the first group and the first CSG operation correspond to a first type of implant planning warning and wherein the second group and the second CSG operation correspond to a second type of implant planning warning.
